(19) United States
(12) Patent Application Publication
(10) Pub. No.: US 2012/0034695 A1
(43) Pub. Date: Feb. 9, 2012

(54) TISSUE/CELL CULTURING SYSTEM AND RELATED METHODS

(76) Inventors: Palaniappan Sethu, Louisville, KY (US); Guruprasad A. Giridharan, Louisville, KY (US); Sumanth Prabhu, Louisville, KY (US); Rosendo Estrada, Louisville, KY (US); Mai-Dung Nguyen, Louisville, KY (US)

(21) Appl. No.: 13/174,034

(22) Filed: Jun. 30, 2011

Related U.S. Application Data
(60) Provisional application No. 61/360,203, filed on Jun. 30, 2010.
(60) Provisional application No. 61/454,152, filed on Mar. 18, 2011.

(51) Int. Cl. C12N 5/071; C12N 5/077; C12M 1/00

(52) U.S. Cl. 435/401; 435/289.1

(57) ABSTRACT

A system for culturing cells and/or tissue includes a tissue/cell culture chamber including a tissue/cell culture membrane, at least one collapsible valve fluidly coupled with the tissue/cell culture chamber, a pump fluidly coupled with the tissue/cell culture chamber, and a flow loop including the pump, chamber, and collapsible valve fluidly coupled together.

### CROSS-REFERENCE TO RELATED APPLICATIONS

This patent application claims the benefit of priority of U.S. Application No. 61/360,203, filed Jun. 30, 2010 and 61/454,152, filed Mar. 18, 2011, which applications are herein incorporated by reference.

### STATEMENT OF GOVERNMENT SUPPORT

This invention was made with government support under Grant #0814194 awarded by the National Science Foundation, and Grant #P30ES014443 awarded by the National Institute of Health. The government has certain rights in the invention.

### TECHNICAL FIELD

The present disclosure relates to a tissue/cell culture model.

### TECHNICAL BACKGROUND

Cardiovascular disease (CVD) is the leading cause of death in the United States and claims more lives each year than the next four leading causes of death combined. Understanding the molecular basis of manifestations of CVD such as myocardial infarction, ischemia, hypertension, cardiomyopathy, heart failure etc, requires multi-scale, multi-level approaches. Analysis of isolated cardiac cells including myocytes, smooth muscle cells, endothelial cells and fibroblasts, free from connective tissue and contaminating cell populations enables assessment of sub-cellular mechanisms and signaling processes in great detail that is typically not possible using intact tissue. However, replicating the mechanical environment of intact cells is a challenging task fraught with intrinsic difficulties, as available in-vitro techniques fail to adequately mimic the in-vivo environment. The myocardium experiences passive stretching and pressure build-up during filling (preload) and actively generates mechanical force during ejection (contraction) against variable afterload. Cells in cardiac tissue therefore are under constant physical stimulation and rely on conversion of these cues into intracellular signals to control cell phenotype and muscle mass during conditions such as hypertrophy. Different from other cell types in the body, cardiomyoctes experiences continuously the pressure changes due to loading and unloading conditions of the heart, causing the cells to contract and relax frequently.

Despite advances in isolation and culture techniques, the current state of cardiovascular in-vitro models is predominantly based on glass slides or tissue culture dishes under static conditions. Most studies utilize isolated cells maintained in two-dimensional culture forming randomly oriented cell-extra-cellular matrix (ECM) attachments.

The blood vessel is an active integrated organ consisting of endothelial cells (ECs), smooth muscle cells (SMCs) and fibroblasts in a highly interactive signaling environment. The vasculature is capable of sensing both mechanical and biochemical signals and transducing theses signals into intracellular cues to influence organization of tissue structure and function. The pulsatile flow of blood generates time-varying biomechanical forces in the form of pulsatile pressure, stretch and shear stress that act on ECs (particularly arterial ECs) that form the innermost layer of the blood vessels. Pathological conditions affecting the cardiovascular system influence mechanical loading patterns and subject ECs to various types of biomechanical forces. ECs respond to these biomechanical force signals via conserved response mechanisms which include inflammation and tissue remodeling through events like proliferation, cell migration, apoptosis, cell-cell and cell-extracellular matrix (ECM) reorganization (altering vascular tone and permeability).

Normal large diameter blood vessels experience average shear stress in the range of ˜10-30 dynes/cm² with peak shear stress values between 40-75 dynes/cm². ECs cultured under these conditions in vitro show an elongated and aligned phenotype, low EC turnover, reduced oxidative stress, low accumulation of low density lipoprotein (LDL), low DNA synthesis and minimal expression of adhesion/inflammatory molecules. Low shear stress and disturbed oscillatory flow that typically occurs in regions of branching or bifurcation results in polygonal and randomly oriented phenotype, high EC turnover, increased accumulation of LDL, increased oxidative stress, increased DNA synthesis and higher expression of proinflammatory adhesion molecules. Functionally, laminar and pulsatile laminar shear stress have been shown to cause vasodialation via upregulation of vasodialators like nitric oxide (NO)³ and nitric oxide synthase (NOS)³ as well as increase in production of Cu/Zn superoxide dismutase which results in dismutation of superoxide which has been implicated in inducing endothelial dysfunction via a several mechanisms including NO inactivation.

Evaluation of stretch on ECs and SMCs has been accomplished in vitro by culture on thin flexible substrates. Further control over the direction and magnitude of stretch can be achieved by controlling the type of stretch via uniaxial, biaxial and circumferential loading. ECs and SMCs in the blood vessel are subject to uniaxial stretch (hoop stress) as a consequence of pulsatile pressure in a direction perpendicular to flow of blood. Therefore the immediate response of ECs to uniaxial stretch is alignment in a direction perpendicular to the direction of stretch (in the direction of flow in blood vessels) and activation of stretch activated ion channels for Ca²⁺ ion transport. In vitro studies demonstrate that short term stretch results in modulation of vessel tone through synthesis of superoxide known to play a role in vasoconstriction whereas prolonged exposure to stretch increases expression of vasodialators NO and NOS¹. Additionally, increase in expression of endothelin 1 (ET-1), a vasoconstrictor implicated in the progression of atherosclerosis, has been demonstrated in vitro.

Unlike shear stress and stretch, relatively few in vitro studies have focused on the direct effects of pressure on EC structure and function. This can be attributed to the assumption that pulsatile pressure from blood flow causes the blood vessel to stretch and therefore the overall effect of pressure manifests itself primarily in the form of stretch. However, evaluation of pressure on ECs in vitro shows that pressure alone in the absence of stretch results in increased EC proliferation, cytoskeletal reorganization and synthesis of ECM proteins. Further, hydrostatic pressure indirectly affects cultured EC monolayer permeability via NO¹⁷ and Ca²⁺ signaling. Elevated hydrostatic pressure mediates an increase in Ca²⁺ transport into cultured ECs, which in turn reduces the permeability of the cultured EC monolayer.

A majority of in vitro studies have limited investigations to evaluating the effects of isolated modes of mechanical stimulation. Despite the generation of large quantities of data regarding EC stress response which has led to significantly improved understanding of EC signaling mechanisms, the fact remains that the response of ECs to individual stimuli is very different from in vivo-like simultaneous and coordinated stimulation.

### SUMMARY

A system allows in vitro hemodynamic stimulation of cardiomyocytes by directly coupling cell structure and function with fluid induced loading. Cells are cultured in a small tissue/cell culture chamber on a thin flexible membrane. Integrating the cell culture chamber with a pump, pulsatile valve and an optional adjustable resistance element in series allow replication of various loading conditions experienced in the heart.

A system for culturing cells and/or tissue includes a tissue/cell culture chamber including a tissue/cell culture membrane, at least one collapsible valve fluidly coupled with the tissue/cell culture chamber, a pump fluidly coupled with the tissue/cell culture chamber, and a flow loop including the pump, chamber, and collapsible valve fluidly coupled together.

In another embodiment, a system includes a tissue/cell culture chamber including a tissue/cell culture membrane, at least one collapsible valve fluidly coupled with the tissue/cell culture chamber, a pump fluidly coupled with the tissue/cell culture chamber, and a flow loop including the pump, chamber, and collapsible valve fluidly coupled together.

In an embodiment, a method includes pumping fluid into a tissue/culture chamber with a pump, closing a collapsible valve and raising pressure in the chamber, and stretching a tissue/cell culture membrane, where the membrane has cultured cells.

In another embodiment, a system includes a tissue/cell culture platform including at least a tissue/cell culture chamber, a tissue/cell membrane having cultured cells thereon, a pump fluidly coupled with multiple chamber tissue/cell culture platform, a pressure generator communicatively coupled with tissue/cell culture platform, and a pressure sensitive valve fluidly coupled with the tissue/cell culture chamber that allows ejection of fluid from the tissue/cell culture chamber based on a predetermined pressure.

In an embodiment, a system comprises a flow loop including one or more tunable elements and a pump, a tissue/cell culture chamber within the flow loop, the tissue/cell culture chamber having a tissue/cell membrane, the tissue/cell membrane having cultured cells thereon. The tissue/cell membrane is deformable in response to pressure buildup within the tissue/cell culture chamber and assumes a concave shape. The system further includes a pulsatile chamber adapted to imitate a heart.

A method includes introducing fluid into a tissue/cell culture chamber of a tissue/cell culture platform chamber system, the system including a tissue/cell culture platform including the tissue/cell culture chamber, a tissue/cell membrane having cultured cells thereon, a pump fluidly coupled with multiple chamber tissue/cell culture platform, a pressure generator communicatively coupled with tissue/cell culture platform, and a pressure sensitive valve fluidly coupled with the tissue/cell culture chamber that allows ejection of fluid from the tissue/cell culture chamber based on a predetermined pressure. The method further includes deflecting the tissue/cell membrane and stretching the cultured cells, applying external pressure including compressing volume and increasing pressure in the tissue/cell culture chamber, and ejecting fluid from the tissue/cell culture chamber when pressure therein exceeds a predetermined value.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1A is a view of a system as constructed in accordance with at least one embodiment.

FIG. 1B is a view of a collapsible valve as constructed in accordance with at least one embodiment.

FIG. 1C is a view of a culture chamber as constructed in accordance with at least one embodiment.

FIG. 1D is a view of a supporting layer with circular post as constructed in accordance with at least one embodiment.

FIG. 1E is a view of a portion of the system as constructed in accordance with at least one embodiment.

FIG. 2A is a system used for estimation of strain values at different pressures, as constructed in accordance with at least one embodiment.

FIG. 2B is a plot of % strain v. pressure for different membrane thicknesses.

FIG. 3A is a system used for pressure characterization in accordance with at least one embodiment.

FIG. 3B is a view of micro pressure tip catheter inserted inside the cell culture chamber and measurements were directly recorded, in accordance with at least one embodiment.

FIG. 4 is a table of μCCCM values and waveforms in accordance with at least one embodiment.

FIGS. 5A-5D illustrate shear stress simulations for an inlet flow velocity of 0.024 m/s for a device with varying well depth.

FIGS. 6A-6D illustrate microscopy images of H9C2 cells in accordance with at least one embodiment.

FIG. 7 illustrates a volume and pressure diagram of a pump cycle of the heart.

FIG. 8 illustrates a relationship between pressure and volume during the pump cycle of the heart, including a cardiac cycle diagram.

FIG. 9 illustrates a schematic of a system flow loop as constructed in accordance with at least one embodiment.

FIG. 10 illustrates a cross section of a μCCCM as constructed in accordance with at least one embodiment.

FIG. 11A illustrates a cross section of a μCCCM as constructed in accordance with at least one embodiment.

FIG. 11B illustrates a cross section of a μCCCM as constructed in accordance with at least one embodiment.

FIG. 11C illustrates a cross section of a μCCCM as constructed in accordance with at least one embodiment.

FIG. 11D illustrates a cross section of a μCCCM as constructed in accordance with at least one embodiment.

FIG. 12 is a summary of critical values for pressure, flow, strain, and shear stress using the system.

FIG. 13 illustrates a replication of pressure, flow, shear and strain waveforms associated with normal, heart failure, hypotension, hypertension, tachycardiac, and bradycardiac conditions.

FIG. 14 illustrates a flow diagram as constructed in accordance with at least one embodiment.

FIG. 15A illustrates a schematic of a system flow loop as constructed in accordance with at least one embodiment.

FIG. 15B illustrates a perspective view of a tissue/cell culture chamber as constructed in accordance with at least one embodiment.

FIG. 15C illustrates a cross-sectional view of the tissue/cell culture chamber of FIG. 15B.

FIG. 16A illustrates laser induced fluorescence images of a channel of the system as constructed in accordance with at least one embodiment.

FIG. 16B illustrates a plot of strain as a function of applied pressure measured using laser induced fluorescence.

FIG. 17 illustrates a comparison of pressure, flow strain, and shear waveforms from a parallel plate system and an ECCM system.

FIG. 18A illustrates a replication of pressure, flow, shear and strain waveforms associated with normal condition.

FIG. 18B illustrates a replication of pressure, flow, shear and strain waveforms associated with heart failure condition.

FIG. 18C illustrates a replication of pressure, flow, shear and strain waveforms associated with hypertension condition.

FIG. 18D illustrates a replication of pressure, flow, shear and strain waveforms associated with hypotension condition.

FIG. 18E illustrates a replication of pressure, flow, shear and strain waveforms associated with exercise condition.

FIG. 18F illustrates a replication of pressure, flow, shear and strain waveforms associated with bradycardia condition.

FIG. 19 is a summary of critical values for pressure, flow, strain, and shear stress using the system.

FIG. 20A illustrates an immunofluorescence microscopy following cell culture using static process.

FIG. 20B illustrates an immunofluorescence microscopy following cell culture using flow only process.

FIG. 20C illustrates an immunofluorescence microscopy following cell culture using the ECCM system.

FIG. 20D illustrates a cell area plot of FIGS. 20A, 20B, and 20C.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope is defined by the appended claims and their equivalents.

A microfluidic cardiac cell culture model (μCCCM) is a system 100 that enables design of experiments where physical loads can be manipulated. Cells cultured under normal conditions can be gradually or instantaneously exposed to loads associated with cardiac dysfunction causing changes in cell structure and function Accomplishing this at the cellular level in vitro provides the opportunity to probe in great detail the role of specific molecular mediators involved in the signaling associated with manifestations of cardiovascular disease. Cells can be evaluated using microscopy directly within the μCCCM system 100 or cells or cellular contents can be extracted and evaluated for gene and protein expression. The cell culture medium can also be sampled continuously to monitor signaling through soluble factors. The μCCCM system 100 therefore provides an ideal platform for evaluating the effects of drugs and other adjunctive and conjunctive treatment options for recovery of cardiomyocytes following cardiac dysfunction. In certain cases the use of external support to return cardiac cells to normal physiological loads has shown to be beneficial to recovery. This scenario can also be replicated by seeding dysfunctional cells or returning cells within the μCCCM system 100 exposed to non-physiological loads to normal loading conditions and then evaluating the structure and function of cells.

Various clinical studies have supported the notion that cardiac tissue can be regenerated through transplantation of progenitor and differentiated cell populations. Although existing protocols have not achieved the goal of true regeneration, substantial physiological benefit (repair) currently can be derived from transplanting cells into the infarcted heart. Though a majority of initial hypotheses centered on improvement in cardiac function with transplanted cells augmenting the host myocardium and beating in synchrony, benefits also seem to indicate reversal in ventricular remodeling and reduction in the infarct size through physical reinforcement and paracrine signaling. Tracking the fate of transplanted cells and determining their true role in improving cardiac function is extremely challenging due to the heterogeneity of cells within the cardiac tissue. The μCCCM system 100 can be used to evaluate differentiation or transdifferentiation potential of various cellular populations in vitro where molecular signaling events responsible for regeneration can be discovered. It is interesting to note that various studies show that benefit can be derived from a wide variety of cardiogenic and non-cardiogenic cell types including adult cardiomyocytes, skeletal myoblasts, smooth muscle cells, fibroblasts, endothelial progenitors, mesenchymal stem cells, hematopoietic stem cells, other marrow populations, resident myocardial progenitors, and embryonic stem cells.

The μCCCM system 100 can be used to culture various cells within the cardiac tissue including cardiomyocytes, smooth muscle cells and cardiac fibroblasts, or cardiac endothelial cells. This system is also capable of co-culture of two or more cardiac cell types to accomplish a more physiologically relevant cell culture model. The primary difference between cardiac cells in the heart and the μCCCM system 100 is the fact that cyclic pressures and mechanical loads are achieved by manipulating the pneumatic valve, downstream resistance, and fluid flow as opposed to cardiomyocyte contractions. Despite this, the μCCCM 100 system enables cardiac cell types to experience physiologic levels of pressures and loads within the native ventricle.

The μCCCM system was developed as an in vitro model of the left ventricle, and accurately replicates aspects of physical loads and maintains a synergistic balance between pressure, stretch and frequency. Pressure, stretch and shear corresponding to normal and abnormal loading conditions have been accurately replicated. Finally, cells were cultured within this system to demonstrate proof-of-concept of the ability of the μCCCM system to sustain cell culture.

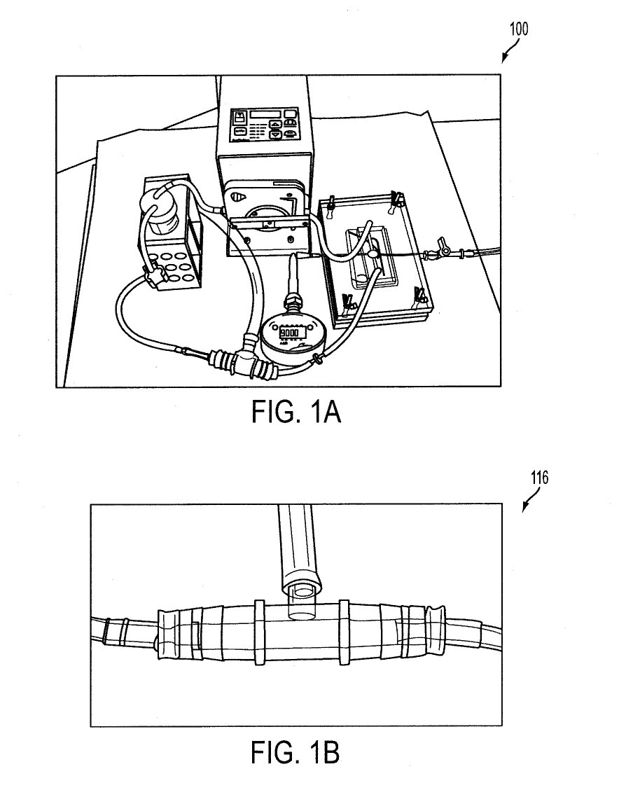

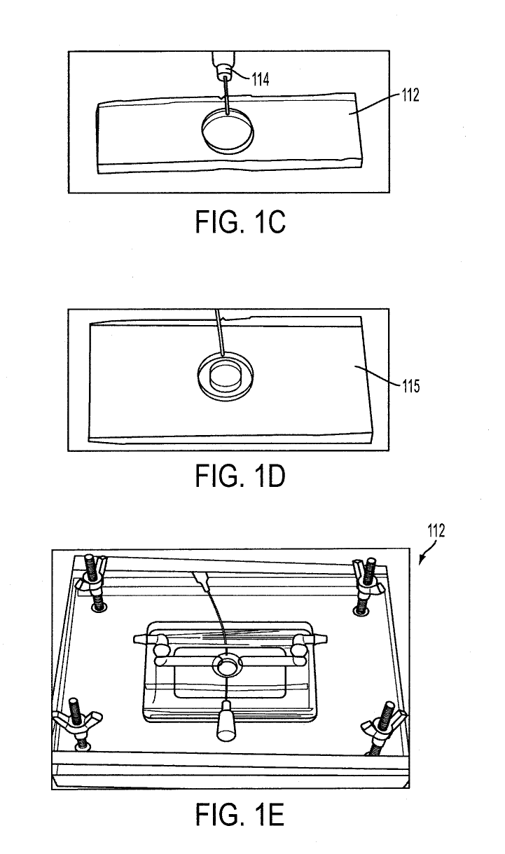

In an embodiment, referring to FIG. 1A, the microfluidic cardiac cell culture model (μCCCM) system 100 is fabricated using standard soft-lithography techniques that include, in an example, a small (1 cm diameter) cell culture chamber on a thin membrane. Continuous circulation of culture medium through the flow loop is maintained using a peristaltic pump 110. Downstream of the chamber 112 is a collapsible valve 116 actuated in a pulsatile fashion using a pressure generator 118. Closure of this collapsible valve 116 leads to pressure build-up in the chamber 112 which in turn also leads to stretching of the thin membrane 114 on which cells are cultured mimicking diastolic preloading in the heart. To ensure uniformity, a post 115 is placed beneath the thin membrane 114 such that during stretch, the portion of the membrane 114 on which cells are cultured experiences uniform strain and the edges experience larger non-uniform strain. Further to influence outflow resistance, a tunable hemostatic valve 120 is placed downstream of the collapsible valve 116 to mimic afterload. This system 100 is programmable and can accommodate a wide range and different combinations of operating parameters. Fluid transport and shear stress can be controlled by setting the flow rates of the pump. Pressure buildup and strain can be controlled via a combination of factors including fluid flow rate and operation of both valves. Strain is also a function of the thickness of the membrane on which cells are cultured. The μCCCM system 100 mimics the native heart where changes in one or more variables have a cascading effect the entire system. For example, increasing the outflow resistance by manipulating the hemostatic valve 120 indicates a system with high afterload. This in turn results in an increase in base pressure and baseline strain within the cell culture area mimicking conditions experienced during aortic stenosis or hypertension.

The following is a discussion of an example of tissue/cell culture platform 112 fabrication. It should be noted that other thicknesses, speeds, times, sizes, etc. are contemplated herein. The tissue/cell culture chamber 112 was fabricated using a two-step process. First, a thin membrane of PDMS (Dow Corning, Midland, Mich.) was fabricated by mixing the pre-polymer with the cross-linking agent in a ratio of 10:1 and spinning this mixture on a silicon wafer at speeds ranging from 200 rpm-2000 rpm on a spin-coater (Laurel Technologies, North Wales, Pa.) to obtain membranes of different thicknesses. Following spinning the silicon wafer was transferred to an oven (Fisher Isotemp, Florence, Ky.) and the PDMS was allowed to cure for three hours. Separately, the PDMS pre-polymer and cross-linking agent mixture were molded into a 3 mm thick layer in a petri-dish and cured in the oven for three hours. Once the cross-linking was complete a 7.5 mm×2 cm piece of PDMS was cut out from the 3 mm thick layer and a 1 cm diameter hole was punched using a cork borer and bonded irreversibly to the thin PDMS layer on the silicon wafer following exposure to oxygen plasma in a reactive ion etcher (March Instruments, Concord, Calif.). This formed the cell culture chamber. Another 7.5 cm×2 cm piece was also cut out and a 1 cm diameter hole was cut from the piece. The 1 cm diameter piece was taken and the diameter was reduced to 7 mm using a different cork borer to produce the post for uniform strain. The 7 mm diameter post was assembled concentrically within the punched hole and the cell culture chamber was assembled on top of this assembly. This arrangement was sandwiched between two polycarbonate plates. The top polycarbonate piece contains an inlet and outlet channel micro machined using an end-mill cutter and contains connections for inlet and outlet tubing. The two plates are clamped using, for example, four screws.

The following is a discussion of an example fabrication of the collapsible valve 116. It should be noted that other thicknesses, speeds, times, sizes, etc. are contemplated herein. The pulsatile actuated valve 116 was fabricated by enclosing a 3 cm long, 5 mm diameter ARGYLE™ Penrose Tubing which is made of latex with a wall thickness of 500 μm inside of a polypropylene T-junction which is 3 cm long, 2 cm tall and has 1 cm diameter openings. The Penrose tube enters and exits along the top of the ‘T’ and the outlet at the bottom is connected to a programmable pressure sensor. The inlet and outlet where the Penrose tube is inserted are sealed to ensure pressure build up within the T-junction and cause collapse of the latex tube. The volume of the Penrose tube within the T-junction was determined based on the volume of the cell culture chamber that needs to be evacuated to ensure return to baseline stretch and pressure. The valve was pneumatically actuated using a programmable pneumatic driver (LB Engineering, Germany).

The following is a discussion of example tissue or cells that can be used or grown in the system 100. The tissue/cells can include, but are not limited to cardiac cells, endothelial cells, smooth muscle cells, fibroblasts, or any cell associated with a vessel. In another example, H9c2 cells are used. H9c2 cells (ATCC, Manassas, Va.), an embryonic cardiomyoblast line, were maintained in culture using high glucose Dulbeco's Modified Eagle's Medium (DMEM) supplemented with 10% fetal bovine serum (FBS) and 1% penicillin-streptomycin. Prior to seeding cells within the device, the devices were sterilized and fitted with a stencil to ensure seeding was limited to a circular area (7 mm in diameter). The devices were treated with 50 mg/ml of fibronectin for 24 hrs at 370 C to promote cell adhesion. Following washing with sterile 1× phosphate buffered saline (PBS), H9c2 cells were seeded at a density of 5×105 cells/ml. Following seeding, the cells were allowed to attach and spread for 4 hours. Following this, the medium was replaced with fresh medium and cells were cultured for 24 hours. The stencil was removed and cells were either cultured under static conditions (control) or under perfusion and pulsatile stretch.

The system 100 was assembled and the pressure build-up within the chamber was characterized for the following clinically relevant experimental conditions—(1) normal, (2) heart failure, (3) hypertension, (4) hypotension, (5) tachycardia, and (6) bradycardia. To accomplish pressure monitoring within the chamber, a small hole was punched using a 24 gauge syringe needle. An 18 gauge needle was then inserted into the hole to form a tight seal and attached to a hemostatic valve using luer connectors. A high-fidelity pressure catheter (Millar Instruments, Boston, Mass.) was inserted through a hemostatic valve and syringe needle such that the pressure sensing element resided in the center of the chamber. The chamber pressure data were signal conditioned and analog-to-digitally converted at a sampling rate of 400 Hz in 30-second recording epochs and stored for digital analysis with a clinically approved Good Laboratory Practice—compliant data acquisition system. The transducers were pre- and post-calibrated against known standards to ensure measurement accuracy. The chamber pressure waveforms were analyzed by using a Hemodynamic Evaluation and Assessment Research Tool program developed in Matlab (MathWorks, Natick, Mass.).

Strain developed in the thin membrane was experimentally evaluated. The membrane for these experiments was modified by embedding 6 μm diameter beads (Duke Scientific, Palo Alto, Calif.). The setup was similar to the pressure characterization except a digital pressure gauge replaced the pressure catheter. Also, the outlet was sealed. The entire setup was placed under a microscope and an image was captured using a CCD camera. Using a syringe, fluid was pumped into the chamber to generate a fixed pressure which resulted in stretching of the membrane and induce a change in the relative position of the beads. Another image was captured using the CCD camera and the relative displacement of the beads under a particular pressure was calculated by comparing the image to the control image using Metamorph Software and the strain and % strain values for different pressures and membrane thicknesses were computed.

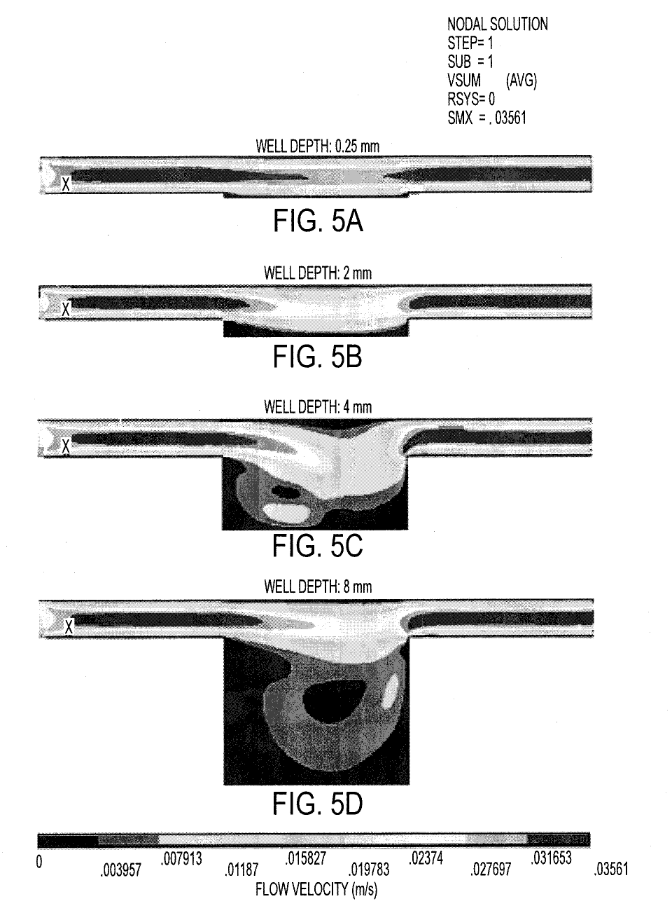

In an example, finite element software ANSYS Academic Teaching Advanced 12.0, FLOTRON module, was used to predict the wall shears stress as a function of channel height and flow rate. The system modeled using a 2D simulation and environment was verified for steady incompressible flows. The physical properties of water were applied to the fluids participating in the simulation (density=1000 kg m−3 and dynamic viscosity=10 Pa s). The inlet fluid flow rates of 8.8 mL/min, was specified at the input, while the outlet was set to a fixed-pressure boundary condition and zero pressure was applied to the outlet. No-slip boundary conditions were applied for the channel and groove walls. The fluid domain was meshed using 2D quadrilateral element, FLUID141, to model steady state fluid. A segregated sequential solver algorithm was used; that is, the matrix system derived from the finite element discretization of the governing equation for each degree of freedom was solved separately. The mesh was refined through mesh sensitivity analyses. At each simulation, the elements showing high velocity gradients were refined, until reaching convergence of sensitive measures of the predicted quantities. FIGS. 5A-5D illustrate shear stress simulations for an inlet flow velocity of 0.024 m/s for a device with varying well depth. As can be seen from the simulations the shear stress is negligible in the region where cells are cultured for all well depths except the 0.25 mm deep well.

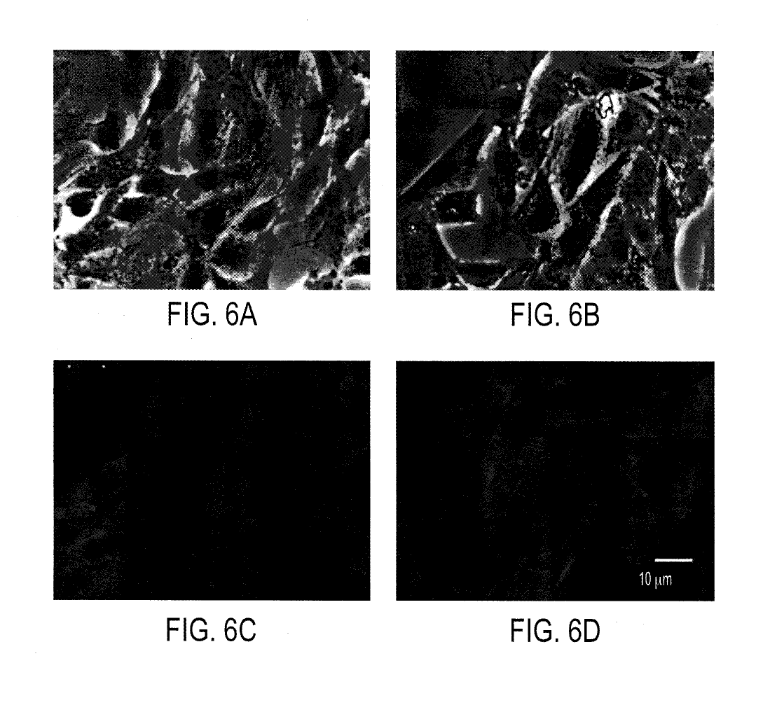

In or order to prepare the cells, H9c2 cells, in an example, were fixed with 4% paraformaldehyde in 1×PBS for 20 min, washed two times with wash buffer (1×PBS containing 0.05% Tween-20 (Fisher Scientific, Fair Lawn, N.J.)) and permeabilized with 0.1% Triton X-100 (Fisher Scientific, Fair Lawn, N.J.) for 5 min at room temperature. For the detection of F-actin, cells were washed two times with wash buffer, blocked with 1% BSA in 1×PBS for 30 min and incubated at room temperature for 1 h with TRITC-conjugated phalloidin (1:100; Millipore, Billerica, Mass.). Light Diagnostics mounting fluid (Millipore, Billerica, Mass.) was added to the cells and cells were examined using a Nikon Eclipse TE2000-U epifluorescence microscope. FIGS. 6A-6D illustrate examples of microscopy images of H(C2 cells. The H9C2 cells were cultured under (left) static conditions and (right) mechanical stimulation. (FIG. 6A, 6B) phase contrast images, (FIG. 6C, 6D) staining with phalloidin to visualize intra-cellular F-actin.

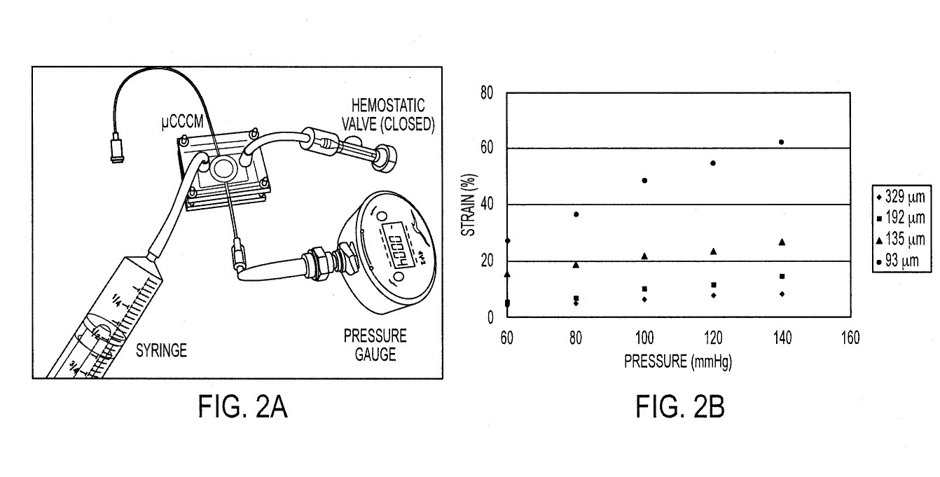

The μCCCM system 100 consists of one or more components: pump 110, tissue/cell culture chamber 112, 114, collapsible valve 116 and the hemostatic valve 120 (FIGS. 1A, 2A) fluidly connected in series to establish a circulation loop. The collapsible valve 116 (FIG. 1B) is responsible for cyclic changes in pressure and strain within the system. FIG. 1C shows the tissue/cell culture chamber 112 with a thin membrane 114 at the bottom for cell culture. This is placed directly over the supporting layer containing a post 130 to ensure uniform stretch (FIG. 1C) and assembled together with fluid flow channels between polycarbonate plates to ensure leak free perfusion (FIG. 1D). The hemostatic valve 120 is downstream of the collapsible valve 116 and is used, for example, to vary afterload. Evaluation of strain was accomplished using the setup shown in FIG. 2B. Membranes of different thicknesses (93, 139, 193, 329 μms) were evaluated for % strain (stretch) at different chamber pressures (60 to 140 mm of Hg). FIG. 2B graphically represents pressure vs. % strain for each thickness. The 139 μm thick membrane resulted in ˜20% strain for normal physiological peak pressures of 120 mm of Hg whereas the 93 μm thick membrane was capable of strains up to 60%.

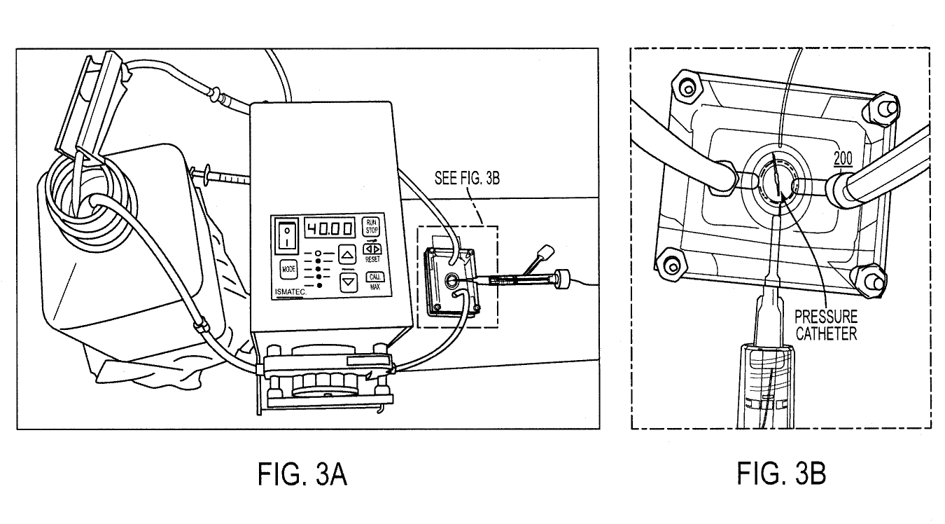

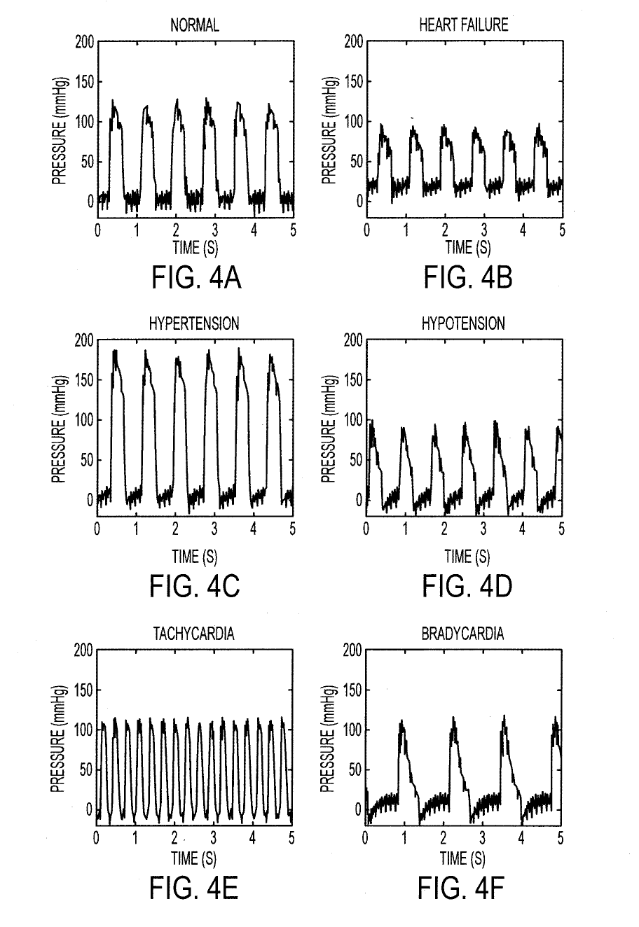

The setup for pressure characterization is shown in FIG. 3. Using this setup the data was continuously recorded for various conditions. The μCCCM system 100 produced a peak pressure of 123 mmHg, an end diastolic pressure of 10 mmHg, at a rate of 75 bpm, and a 40% systolic fraction. Simulated heart failure had a lower peak pressure (95 mmHg) and a significantly higher end diastolic pressure (27 mmHg). Hyper- and hypotension test conditions produced normal end diastolic pressures (8-10 mmHg). Hypertension test condition had a significantly elevated peak pressure (183 mmHg) while hypotension had a significantly lower peak pressure (92 mmHg) compared to normal test condition. Simulated tachycardia had a significantly higher beat rate (200 bpm), lower end diastolic value (3 mmHg), and a higher systolic fraction (55%) compared to normal test condition. Simulated bradycardia had a lower beat rate (46 bpm), and slightly lower systolic fraction (38%) compared to normal test condition. The obtained μCCCM values and waveforms (FIG. 4) closely correlate to human left ventricular waveforms found in literature for experimental test conditions.

To evaluate shear stress within the system, CFD modeling was performed in an example. The depth of the well was varied (0.25, 2, 4, 8 mm) while the inlet flow rate was kept constant ˜0.024 m/s. It can be seen from the simulations that for the 2, 4 and 8 mm depths, fluid flow is not significant and shear stress is ˜0 from the floor of the wells till ˜200 μm above the well (region where cells are cultured). However, for the 0.25 mm depth shear stress is significant (˜10 dynes/cm2) at 200 μm from the wall and below, in the region where cells are cultured within the device. An additional variable which has a major effect on shear stress but was not evaluated due to cascading effects on pressure and stretch is the inlet fluid flow velocity.

In an embodiment, H9C2 cells were cultured in static controls and within the μCCCM system 100 under pressure and stretch representing normal physiological loading. Cell cultured in static controls attain a fibroblast like morphology (FIG. 6A) whereas cells cultured in the μCCCM attained a more rectangular, postage stamp-like morphology (FIG. 6B). Staining with phalloidin showed random orientation of F-actin in controls in comparison to aligned F-actin in cells cultured within the μCCCM system 100 (FIGS. 6C and 6D).

The μCCCM 100 is a system that was designed as an in vitro model of the left ventricle capable of accurately replicating complex in vivo mechanical stresses associated with ventricular loading for a wide variety of clinical conditions. The system 100 was designed such that change in one aspect of loading has a cascading effect on other variables similar to events in vivo. This is made possible in the μCCCM system 100 by integrating one or more of four tunable components in a fluidic circuit. The pump 110 induces flow and the rate of flow can be manipulated to increase/decrease shear stress and control rate of loading. Tissue or cells are cultured on a thin membrane 114 within the tissue/cell culture chamber 112, where the thickness of the membrane plays a role in determining the amount of stretch for a given pressure. The collapsible pulsatile valve 116 has several tunable variables including collapsible volume, pressure used to achieve collapse, frequency and timing at which this pressure is applied and valve open:close ratio (systolic:diastolic). An adjustable hemostatic valve (resistance element) 120 controls the afterload in the circuit. Again, change in one or more of these variables affect the magnitude and duration of pressure and stretch within the system. Cells such as H9c2 cardiomyocytes were successfully cultured within the device. The cells not only survived physiological loads in vitro but the effects of physiological loading were clearly evident when analyzed via microscopy. For example, cells within the μCCCM system 100 established an in-vivo postage stamp-like morphology and showed alignment of F-actin stress fibers in comparison to static controls.

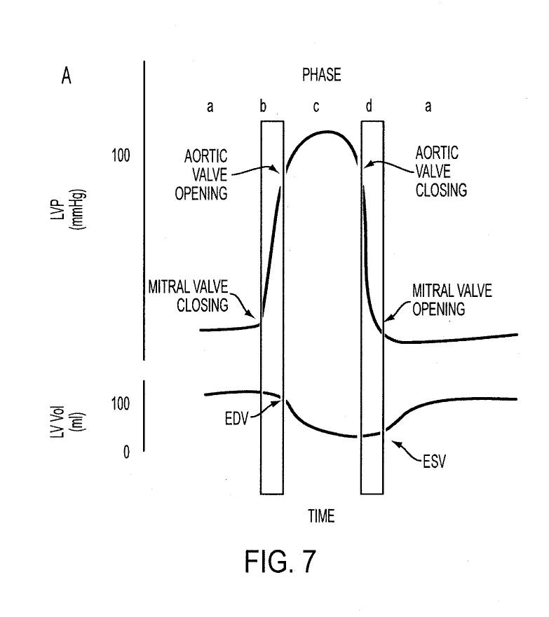

Referring to FIGS. 7-14, system 200 was designed to mimic the functioning of the left ventricle in the heart. In the system 200, the stretching of the membrane is affected directly by the fluid dynamic pressure, in which the stretching increases with the increasing of the pressure.

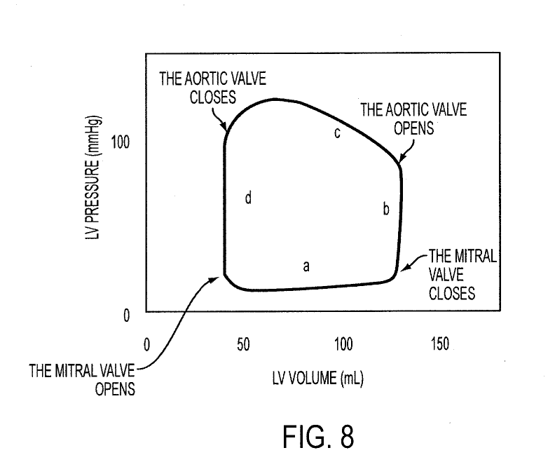

With respect to system 200, understanding the working behavior of the heart will help to study the heart at the cellular molecular level. Due to the heart structure and function, heart muscle cells or cardiomyocytes continuously experience the contractility and pressure changing during the heart pump cycles. Therefore, to manipulating the physiological condition of the heart for the in vitro studies at the cellular level, cardiomyocytes are grown on a flexible membrane that is under pressure changing due to fluid dynamic condition in order to contract and relax. The heart functions as a hemodynamic pump to deliver blood throughout the body through its ability to contract and relax in rhythm. The magnitude and duration of stretch and contractions in cardiac tissue depend on the preload and afterload conditions of the heart cycle. Especially in the left ventricle, preload refers to the condition where the heart can be able to relax from the previous contraction. During this preload phase, the mitral valve opens to eject the blood from left atria to left ventricle. Since the heart is relaxing, filling of blood inside the ventricle causes the stretch of the heart wall. Therefore, this preload condition is called the diastolic phase. In this phase, both of the blood and the ventricle volumes increase significantly but the pressure in this chamber changes slightly from ˜4 mmHg to 10 mmHg. The afterload begins when the heart starts to contract. At this moment, both atrial valve and aorta valve are close, causing the isovolumetric contraction which significantly increases the pressure inside the ventricle until it overcomes the aorta pressure. (FIGS. 7-8) Once the ventrical pressure overcomes the aorta pressure, the aortic valve opens and the blood inside the left ventricle is ejected into the aorta via the contractile force. Afterload refers to the resistance in the system that needs to be overcome to pump blood out of the ventricle. Once the blood is injected to the aorta, the ventricle begins to relax. The ventricular pressure at this moment is lower than the aorta pressures, causing the aorta valve closed. However, the mitral valve hasn't opened yet. As a result, the expanding of the ventricle without blood filling drops the pressures rapidly to the base line which is around 4 mmHg. This cycle is repeated.

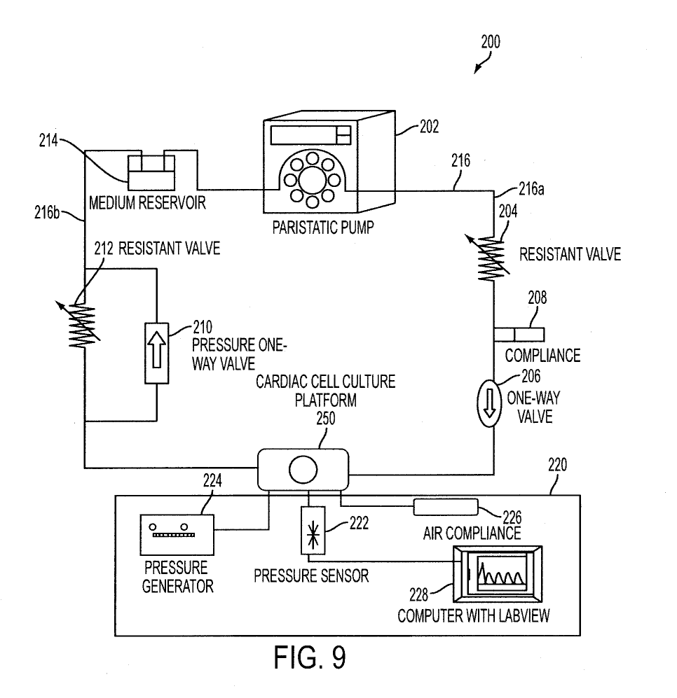

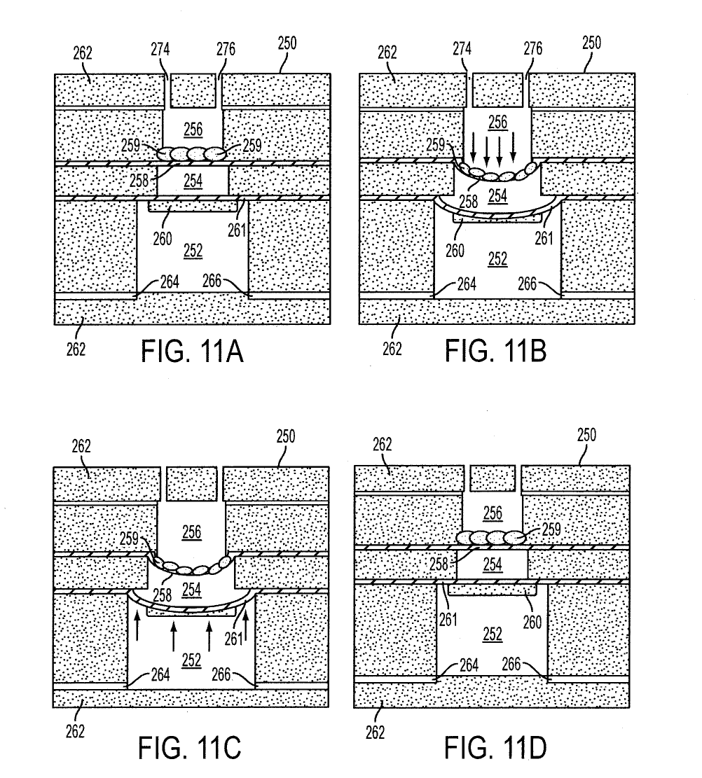

In the system 200, shown for example in FIGS. 9 and 11, the stretching of a membrane occurs during the loading time while the pressure is kept around 0-10 mmHg (See FIGS. 11A, 11B). The pressure of the cell culture chamber rises up when the membrane is contracted. With this model, the relationship between pressure and blood filling volume was established similarly to the physiological condition. The system 200 can include a peristaltic pump 202, a tissue/cell culture platform 250, one way spring pressure valves 210, one way valve 206, one or more tunable resistant valves 204, 212, and a compliance element 208 along with tubing 216, which interconnects the components. The system is controlled by a control module 220, which can include a pressure generator 224 and a pressure sensor 222 connected to a computer system 228, such as a computer system with LabView 9.1 Software program.

The tissue/cell culture platform 250 is composed of multiple chambers, such as a first chamber, a second chamber, and a third chamber, as shown in more detail in FIGS. 11A, 11B, 11C, and 11D. In an embodiment, the multiple chambers include a pneumatic chamber 252, an oil chamber 254, and a tissue/cell culture chamber 256. In a further option, the system 200 includes two or more membranes 258, 261, such as a first membrane and a second membrane. In one example, the first membrane includes a tissue/cell seeding membrane 258 that has tissue/cells 259 thereon. The tissue/cells 259 can include, but are not limited to tissue, smooth muscle tissue, cardiac cells, endothelial cells, smooth muscle cells, fibroblasts, or any cell associated with a vessel.

The second membrane is a membrane that includes a rigid portion. For instance, the second membrane is a post membrane 261 that has a post 260 coupled therewith. In an embodiment, the post membrane 261 has two opposite substantially planar sides, and the post 260 is coupled with one of the sides of the membrane 261. The post 260 is more rigid than the post membrane 261 and located on a lower portion of the membrane 261. This assists in preventing the post membrane 261 from deflecting in a second direction, past the original position. In a further option, the tissue/cell culture platform 250 is disposed adjacent to one or more outer platforms 262, such as a top or bottom platform, which further accommodate inlets and outlets for the platform 250.

There are some advantages of having two membranes instead of one cell culture membranes or having the post attached directly to the cell membrane. First, without the rigid post, the cell culture membrane, once pushed up by pneumatic pressure, would curve up beyond the original position. The post prevents this movement. When the rigid post reaches to the rigid edges of chamber, it stops moving up and the membrane attached to this post cannot move further. Second, if the post were bonded directly to the cell culture membrane, the area bonding between the post and the membrane would become inflexible. It could not be either stretched nor contract, which affects the cells growing on this area and creates an error for the result. If the attaching area is small (<1 mm), the bonding area on the cell membrane can be neglected. However, due to the weight of the post, the cell culture membrane will become un-even especially at the center, which is lower than the other area. In addition, due to the small attaching area, once the post was pushed up, large vibration on the post will create the vibration on the membrane. Third, it is more time consumed and more working steps are needed if the post is bonded to the cell membrane. It must be made for every running experiment, which creates more challenging for other researchers or collaborators who want to use this system but cannot access to the clean room facility or are not familiar with the bonding technique. To solve all these problems, a 1.6 cm glass post is bonded to another membrane and the bonding area is covered fully on one side of the flat post. To prevent the gas permeation through the PDMS (PDMS is gas permeable), Pyrylene is coated only on this membrane. This post membrane is reusable many times until it wears out.

The third chamber, or the bottom chamber is, in an embodiment, a pneumatic chamber 252 which can be pressurized. For instance, air pressure is either added or withdrawn via the pressure generator. Layered on top of the third chamber is the second chamber, such as an oil chamber 254. Between the third chamber and the second chamber is the second membrane, such as the post membrane 261. The second chamber is, in an embodiment, an oil chamber 254 that is filled with oil. The oil chamber 254 is located between the post membrane 261 and the cell culture membrane 258, where the cells are seeded. On the top of the cell membrane is the tissue/cell culture chamber where the cell culture medium is exchanged continuously via the peristaltic pump. The cell culture membrane 258 serves as the floor of the of the cell culture chamber, where the tissue/cell are within the tissue/cell culture chamber 256. The multiple chambers and membranes are layered and then placed between outer platforms 262, such as, but not limited to, two one-inch Plexiglass platforms. The bottom platform has the air inlet 264 and outlet 266 pathway connecting to the pneumatic chamber 252, the pressure generator 224 and optionally a compliance element, such as a balloon. The balloon is used to reduce the noise of the pressure waveform when the air is vacuumed out of the system. The top platform has the inlet 274 and outlet 276 for the cell culture medium.

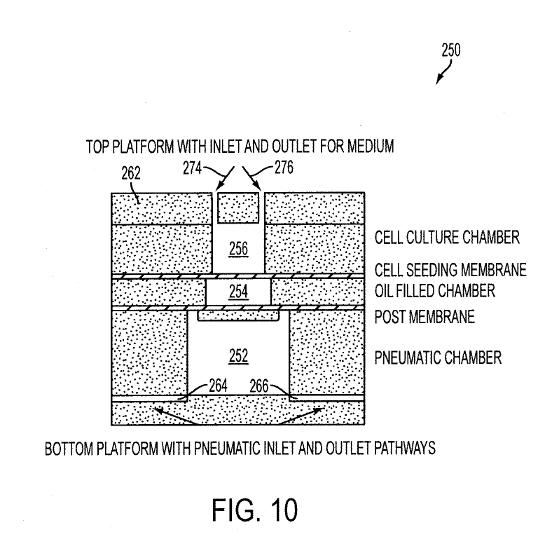

Referring to FIGS. 9 and 10, the system 200 has a first pathway and a second pathway, where each pathway is coupled with one or more components. In an embodiment, the system 200 includes two pathways: a fluid pathway and pneumatic pathway. For the fluid pathway, system 200 is a closed system that mimics the in vivo cardiovascular circulation system. A continuous circulation of culture medium is maintained using the peristaltic pump 202. The peristaltic pump 202 is used to load the medium to the system 200 generating the loading condition. A one-way valve 206 is inserted along the inlet tubing 216a while the pressure one-way valve 210 is connected in parallel to the outlet tube 216b to control the pressure inside the tissue/cell culture chamber 256 as well as the fluid flow direction. The valves 206, 210, such as tunable valves, are also used to increase fluid resistance, and with the pressure generator 224, to generate a pressure inside the tissue/cell chamber 256. The compliance element 208 is used at the inlet to reduce the noise of the pressure waveform due to the loading condition. The compliance element 208 also helps to bring down the pressure to the zero level after the contracting occurs. A pressure sensor 222 is connected to the tissue/cell culture chamber 256, and a pressure profile of the tissue/cell culture chamber 256 is recorded.

The pneumatic pathway is used mainly to push the membranes 258, 261 back to their original position after stretching down and to control the frequency of the stretching. Air from the air source is pumped to the pneumatic chamber via pressure generator at the inlet 264 with a desired pressure, rate, and percentage of the preload phase in a cycle. In normal condition, the pressure applied is 120 mmHg at a rate of 80 beat per minute (bpm) and 40% of the cycle is diastolic phase. At the outlet 266, a pneumatic compliance is used to capture the outlet air and reduce the noise background of the pressure signal. Then, during the systolic phase, the pressure generator vacuums the air inside the pneumatic chamber and the compliance. Due to no pressure applied from the bottom but only from the top, membrane begins to stretch down again during this phase.

During use of the system 200, cell culture medium is loaded into the tissue/cell culture chamber 256, creating a pressure inside the cell and causing the cell membrane 258 to stretch downward to the position shown in FIG. 11B (toward a first direction) from the position shown in FIG. 11A. This creates the loading condition of the heart. At this time, the cells 259 on the membrane 258 are in the relaxed condition. On the bottom side of the cell culture membrane 258, the oil filled inside the oil chamber 254 serves as bulk material to transfer the stress from this membrane 258 to the post membrane 261, causing the post membrane 261 to move downward vertically (toward the first direction) as the cell membrane stretches downward. Then, the pressure generator 224 inserts air with high pressure (ex. 120 mmHg for the normal condition, or 180 mmHg for the hypertension case) to the pneumatic chamber 252 via pneumatic inlet 264, pushing the circular post 260 up to the original position (away from the first direction) which in turn, pushes the cell membrane 258 back to its original position (away from the first direction). The circular post 260 on the post membrane 261 prevents the membranes 258, 261 from curving up beyond the original position. Once the membrane 258 is in the original position, due to hyper-elastic property of the PDMS, the energy in membrane 258 is restored and the membrane 258 is contracted. The cells 259 attached on this membrane 258 are also contracted and the fluid inside the cell chamber is forced to move out via the outlet 276 as the result of the increasing pressure inside the tissue/cell chamber 256 when the tissue/cell membrane 258 is pushed up to its original position. This condition generates the unloading condition which is similar to the unloading of the heart.

The movement of the cell membrane 258 is in the response of loading and unloading conditions of one full pump circle in the system 200. As shown in FIG. 11A, the first chamber is filled and the preloading starts. Referring to FIG. 11B, passive stretch of the membrane 258 during the fluid loading occurs, the changing of pressure inside the tissue/cell chamber 256 remains low, but the volume increases significantly. In, FIG. 11C, the isovolumetric contraction occurs, and the afterload begins. During afterload, pressure inside the tissue/cell chamber 256 increases significantly while the bottom pressure pushes the tissue/cell membrane 258 up toward the original position. In FIG. 11D, the ejection phase occurs, where the pressure inside the chamber overcomes the resistance from the outlet tubing, the fluid is ejected to the vessel and brings the membrane back to the original position, contracting the cells on the tissue/cell membrane 258.

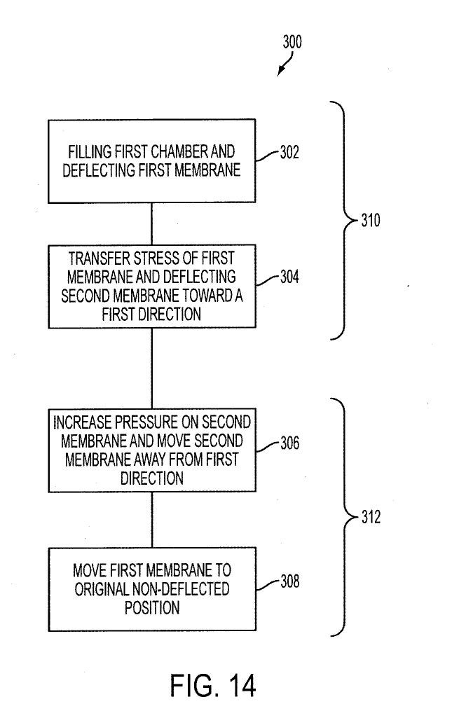

Referring to FIG. 14, a method 302 includes introducing fluid into a tissue/cell culture platform chamber system, the system including a multi-chamber tissue/cell culture platform having at least a first chamber and a second chamber, the first chamber including a tissue/cell culture chamber, the multiple chamber tissue/cell culture platform includes two or more layers of chambers stacked upon one another; a first membrane coupled with the multiple chamber tissue/cell culture platform, the first membrane disposed between the first and second chambers; a second membrane located adjacent the second chamber, a pump fluidly coupled with multiple chamber tissue/cell culture platform, a pressure generator communicatively coupled with multiple chamber tissue/cell culture platform, third chamber, and forming a flow loop. Tissue or cells are disposed on the first membrane. The tissue/cells include one or more of smooth muscle cells, endothelial cells, H9C2 cells or endothelial cells. A first chamber is filled and the first membrane is deflected 302. The method further includes deflecting the first membrane, transferring stress of first membrane to the second membrane and stretching the second membrane toward a first direction 304. This portion 310 mimics the preload portion of the heart cycle. The method further includes increasing pressure on the second membrane and moving the second membrane toward the first membrane away from the first direction 306, and moving the first membrane to original non-deflected position 308. This portion 312 mimics the afterload of a heart cycle.

Other variations for the method include ejecting fluid from the first chamber, or modulating at least one of pressure, stretch, flow or shear stress within the closed loop. In an option, modulating at least one or pressure, stretch, flow or shear stress includes stimulating the tissue/cell culture chamber with one or more pathological conditions similar to heart failure, hypotension, hypertension, tachycardia, or bradycardia. The method further optionally includes pressurizing gas within the third chamber to increase pressure on the second membrane.

The membranes were made using standard soft-lithography techniques. Silicon Elastomeric base Polydimethyl silixane (PDMS) (Sylgard® 184, Dow Corning, Midland, Mich.) was mixed well with its cross-linker at the ratio 10:1, degassed and spun on (10 cm×10 cm) glass slides with 400, or 500 rpm for 30 secs and baked in a 75° C. oven for 2 hrs for curing. Then the thin PDMS layer was peeled off from the glass slide and cut into two (5 cm×5 cm) square membranes. To make the post membrane, a circular glass slide (1.5 mm thick) was bonded at the center of the membrane via the oxygen plasma followed by a 1 min bake on a 95° C. hotplate. This post membrane then coated with a very thin layer of Parylene (0.003 g of Parylene). In an option, the tissue/cell culture membrane 258, after peeled off from the glass slide, was treated with 10% oxygen plasma for 30 sec (100 mTorr, and 100 mmHg) followed by 50 ug/ml of fibronectin solution for overnight. Then the membrane was ready for tissue or cell culturing.

The pneumatic, oil, and cell culture chambers are made with plastic, such as Plexiglass, with the thickness of 1.5, 0.25 and 1.0 cm respectively. A square piece of 5 cm×5 cm was cut out of each piece of plastic and a cylindrical hole was drilled at the way through at the center of each square piece. The diameters of the holes were 1.6, 1.4, and 1.2 cm corresponding to the pneumatic, oil, and cell culture chambers respectively.

For the inlet of pressure sensor, a 5×5 cm PDMS piece (0.5 cm thick) was cut out of the molded piece. A 1.3 cm diameter hole was punched at the center of the PDMS piece. A 15 G needle was used to create an inlet channel. An 18 G needle was used to connect between the cell culture chamber and the pressure sensor via this channel. The platform consists of generally rectangular platforms as discussed above.

To assemble the system 200, the post 260 is coupled with the post membrane 261. An oil, such as, but not limited to olive oil is used to fill the oil chamber 254. The cell culture membrane 258 is gently and slowly laid down on the top of the oil chamber 254 in such a way that there are substantially none or no bubbles trapped inside the oil chamber 254 and no bending of the membrane 258 occurs. The cell culture chamber 156 is set on the top of the cell culture membrane 258. The assembled chambers 256, 254 are gently placed on the top of the pneumatic chamber 252 which is glued in advance to the bottom platform for preventing the air leaks. A PDMS-needle piece is put on the top of the cell culture chamber 256, and a platform 262 is put on the top. All of these components are tightened together via the platforms.

A tubing is to the medium reservoir 214 and to the pulsaltile pump 202, then from the pump 202 to the tunable valve 204, the compliance element 208, and the one-way pressure valve 206 before reaching to the inlet 274 of the tissue/cell culture platform 250. Tubing from the outlet 276 of the tissue/cell culture platform 250, after connected to a one-way spring pressure valve 210 and a tunable valve 212 via T-connector, returns to the medium reservoir 214. After the system 200 is set and the tissue/cell culture chamber 256 is filled with medium, the pressure sensor is connected, and the pressure generator 224 is connected to the pneumatic inlet 264 while the pneumatic compliance is at the pneumatic outlet 266.

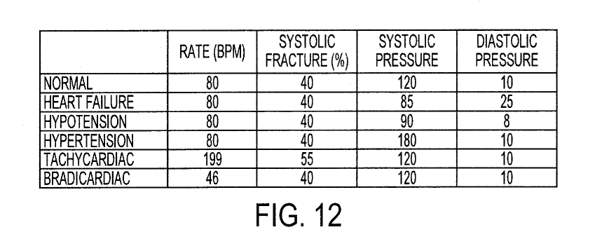

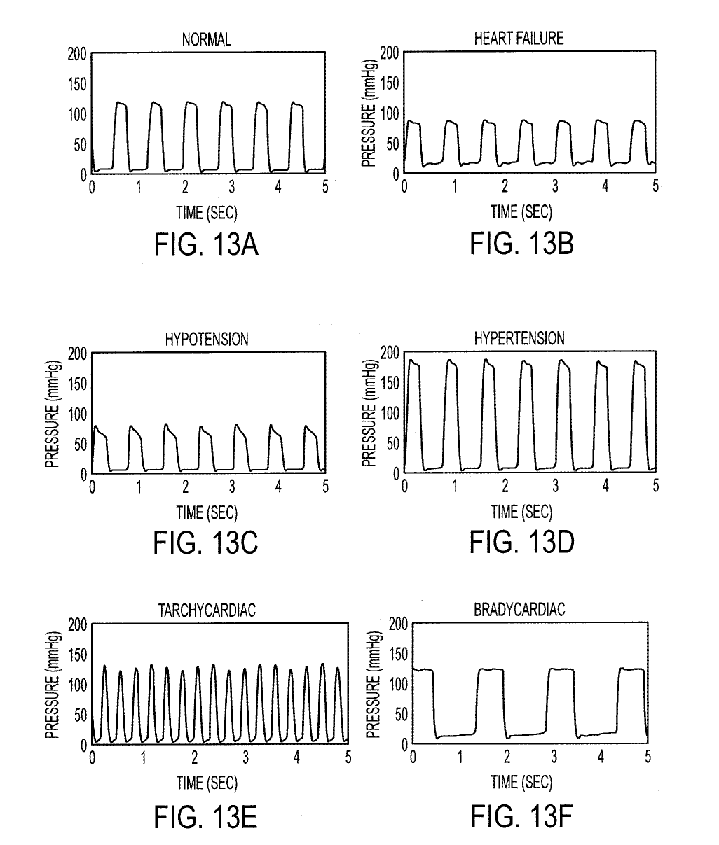

Pressure profiles from different pressure settings based on the different conditions of the heart (normal, heart failure, hypotension, hypertension, bradycardiac, cachycardiac conditions) were recorded, and are shown via a computer program, such as the LabView Program. The pressure waveforms were created using Excel Program. (FIGS. 12 and 13).

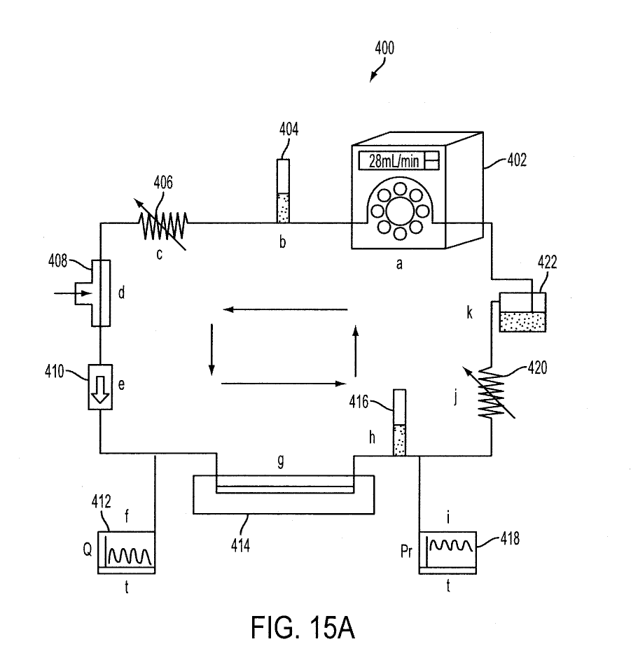

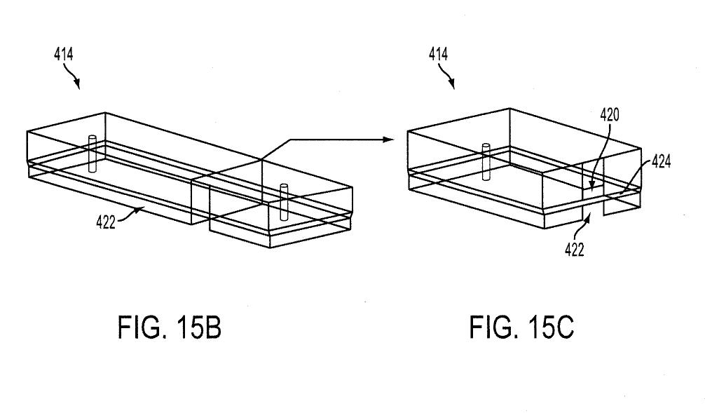

In another embodiment, as shown in FIGS. 15-20 an Endothelial Cell Culture Model (ECCM) System 400 is used to generate realistic pressure, flow, stretch and shear stress profiles associated with normal and dysfunctional cardiac flow, where in vivo mechanical loading conditions can be accurately recreated.

To accomplish realistic in vivo-like biomechanical loading, tissue and/or cells within this system were cultured on a stretchable, thin planar membrane within a rectangular flow channel and subject to constant fluid flow. Under pressure, the thin planar membrane assumes a concave shape and represents a segment of the blood vessel wall. Pulsatility is introduced, for example, using a programmable pneumatically controlled collapsible chamber. Cells, such as, but not limited to, human aortic endothelial cells (HAECs) were cultured within this system under normal conditions and compared to HAECs cultured under static and ‘flow only’ controls using microscopy. Results confirm that cells cultured within the ECCM system 400 are larger than controls, ellipsoidal shape with alignment of actin cytoskeletal filaments and show high levels of expression of β-Catenin indicating an in vivo-like phenotype. The endothelial cells in different locations of the body experience different levels of mechanical stresses and that this system can be used replicate any condition of the different locations within the body.

The platform of the system 400 includes a channel 420, such as a rectangular cell culture channel. The tissue/cells 424 are cultured on a suspended membrane 422, such as, but not limited to, polymeric thin film, inside the rectangular channel 420. The suspended thin film 422 forms a concave shape inside the channel 420 and represents a segment of the blood vessel wall. The membrane 422 in the channel 420 stretches in response to pressure similar to a compliant blood vessel. This system 400 uses adjustable controls, such as analog controls, including compliances, resistances, a collapsible pulsatile chamber and a one-way flow control valve to accurately mimic hemodynamic waveform morphologies associated with normal and pathological conditions. Culture of tissue or cells on a planar surface simplifies cell seeding using standard cell culture techniques and imaging using confocal microscopy. The system 400 is also capable of co-culture with SMCs and compatible with the systems 100, 200 and, for example, can be integrated downstream of cultured cardiomyocytes.

The ECCM system 400 includes a peristaltic pump 402 to induce and manipulate flow through the flow loop, a tissue/cell culture chamber 414 with a membrane 422 that mimics a vessel wall, a pneumatically driven pulsatile chamber, a one-way valve, one or more tunable flow resistance elements to adjust preload and afterload, and one or more tunable compliance elements that represent arterial and venous compliance. The elements of the system 400 form a flow loop through which fluid can flow, and various parameters can be set and/or measured to mimic pathological or normal conditions.

Resistance (preload and afterload) to the tissue/cell culture chamber was accomplished, in an example, using roller clamps (Fisher Scientific, Florence, Ky.). The roller clamps slide over flexible tubing to adjust fluid flow resistance. ‘T’ junctions with an air/carbon dioxide column were used to manipulate arterial and venous compliance elements. Compliances were manipulated by varying the height of the air column in the compliance chambers.

The tissue/cell culture chamber 414, in an option, is a rectangular channel with a compliant thin membrane 422 that serves as the floor of the channel 422. The chamber was fabricated using standard soft lithography techniques. Briefly, a layout of the rectangular channel (2 cm×5 mm) was created using AutoCAD layout software (Autodesk, San Rafael, Calif.) and printed as a darkfield mask on a 5 in×5 in transparency at 20,000 dpi (Fineline Imaging, Colorado Springs, Colo.). A 4 in silicon wafer was coated with 500 μm thick layer of negative photoresist SU-8 100 (Microchem, Newton, Mass.) and patterned using the fabricated mask via exposure to UV light in a mask aligner (Karl Seuss, Garching, Germany) and then developed to produce negative replicas of the desired channel structure. The silicon wafer with the channel patterns was used as a master to mold the cell culture chamber using (poly)dimethyl siloxane (PDMS) (Dow Corning, Midland, Mich.) and cured at 80° C. for 3 hrs in an oven (Fisher Isotemp, Florence, Ky.). Following molding, the channel structures were cut and access holes were punched at the inlet and outlet. Separately, PDMS was spun on a blank silicon wafer at a spin speed of 200 rpm on a spin-coater (Laurel Technologies, North Wales, Pa.) to obtain a 500 μm thick membrane and cured for 3 hrs. The molded channels were assembled along with the membrane and clamped together to form a hermetically sealed perfusion chamber for cell culture.

In an example, the system 400 includes a tissue/cell culture chamber where cells are cultured on a flexible membrane. The cell culture chamber is integrated within a flow loop and various individually addressable and tunable elements that will allow mimicking of virtually any flow and pressure waveform. The flow loop can be assembled as follows: a pump initiates circulation, compliance and resistance elements mimicking the pulmonary compliances and resistances, a pulsatile chamber mimicking the heart, a one-way flow control valve, the cell culture chamber, compliances and resistances mimicking the systemic/aortic compliances and resistance along with pressure and flow sensors. The membrane on which the cells are cultured deforms in response to pressure buildup within the chamber and assumes a concave shape similar to a section of a blood vessel. Cells within the chamber are exposed to pressure, flow, stretch and flow similar to conditions experienced in the body. To generate disturbed or retrograde flow, the one-way flow control valve can be removed.

Flow and pressure measurements were made upstream of the cell culture chamber inlet and downstream of the outlet respectively. Flow measurements were collected real time using an inline, transit time flow probe (Transonics, Ithaca, N.Y.). Pressure measurements were accomplished using an inline pressure sensor (Validyne, San Francisco, Calif.). Signal conditioning was accomplished using transducer amplifiers (Ectron, San Diego, Calif.) and transit-time flow meters (Transonics, Ithaca, N.Y.), and other peripheral conditioners integrated in an instrumentation system compliant with Good Laboratory Practice (GLP) guidelines. Signal conditioned data were low pass filtered at 60 Hz, analog to digitally converted (AT-MIO-16E-10 and LabVIEW, National Instruments, Austin, Tex.) at a sampling rate of 500 Hz.

In conducting stress measurements, Reynolds numbers for flow at flow rates of 50 ml/min in a rectangular channel with cross section area of 5 mm×500 μm were estimated to be ˜80 indicating that the flow is laminar. The Womersley Number (α) which arises in the solution of Navier Stokes equations for pulsatile flow was also determined to evaluate the ratio of pulsatile flow frequency to viscous effect using the following equation (1):

$$α = R \sqrt{\frac{2 π f ρ}{μ}} \qquad ( 1 )$$

where: f is the frequency, ρ is the fluid density, μ is the dynamic viscosity and R is the hydrodynamic radius of the channel structure. Small α values˜1 indicate that the pulse frequency is sufficiently low that a parabolic velocity profile develops during each cycle and is a good approximation to Poiseiulle Flow. Whereas large α value>10 indicates that the velocity profile is flat resulting in plug flow. For this system, α values were relatively low ranging from 1.2-1.8. Accounting for the pulsatile flow the shear stress (τ) was estimated using the following equation (2):

$$τ = \frac{α}{\sqrt{2}} \frac{6 Q μ}{{h}^{2} w} \qquad ( 2 )$$

where: Q is the volume flow rate attained from the inline flow sensor and h and w the height and width of the channels respectively. Following pressure build up, the thin membrane (floor of the channel) expands altering the cross-section of the channel and hence affecting the shear stress. The overall increase in ‘w’ was approximated from strain calculations and used to estimate appropriate shear stress values based on the changes in fluid flow.

In order to measure strain, a Laser Induced Fluorescence (LIF) technique was used to measure the deflection the PDMS membrane within the ECCM system 400 as a function of applied pressure. In the LIF system, a thin light sheet from a 600 kJ/pulse, Nd-YAG laser (NewWave Research, Fremont, Calif.), operating at a wavelength of 532 nm, was directed vertically along the center plane of the channel. The thin laser sheet is created using a number of optics including concave and convex lenses. The entire device was filled with a fluorescently labeled dye: Fluorescein (Sigma Aldrich, St. Louis, Mo.) and the glowing dye at the intersection of the light sheet and the solution was imaged using a high-resolution digital camera (PowerView 4M Plus with 2048 2048 resolution and 12 bits depth, TSI Inc., Shoreview, Minn.), which was mounted on an optical rail attached to the optical table. The glowing dye at the intersection of the PDMS surface and the laser light sheet creates a sharp, clearly visible black-to-white edge in the images. A gradient-based algorithm was used to trace this edge in each image. The resulting PDMS surface profile was obtained in the image coordinate system with units of pixels. In order to transform the PDMS surface profile into physical space, an inverse mapping procedure was employed. Before and after every set of tests, an image of a calibration target, which was placed in the plane of the laser light sheet, was recorded with the camera in the same position and orientation as when PDMS surface profile measurements are performed. The calibration target image was used to map image coordinates into physical coordinates. The origin of the physical coordinates was taken as the center of the intersection of the undisturbed PDMS surface and the calibration target when the applied gauge pressure was zero.

In an option, cells 424 are disposed on the membrane 422. In an option, the cells are HAECs (Human Aortic Endothelial Cells) (Invitrogen, Carlsbad, Calif.). These cells were initially maintained in culture using Medium 200 (Invitrogen, Carlsbad, Calif.) supplemented with Low Serum Growth Supplement (LSGS; Invitrogen, Carlsbad, Calif.) and 1% penicillin-streptomycin. Prior to seeding cells within the device, the PDMS membrane and the channel were exposed to oxygen plasma for 0.5 min and bonded. The devices were treated with 50 mg/ml of fibronectin for 12-18 hrs at 4° C. and for 30 min at 37° C. to promote cell adhesion. Following washing with cell culture medium, HAECs cells were seeded at a density of 5×10⁵ cells/ml. Cells were allowed to attach and spread and after 4 h the medium was replaced with fresh medium. Cells were cultured for 24 h and then maintained under static conditions (control) or laminar flow or under perfusion and pulsatile stretch.

In an example, HAECs were seeded within the ECCM system 400 using a stencil to restrict cells to the middle 1.5 cm long region (where strain is unidirectional and uniform) and maintained in culture until they reached confluence. Once they reached confluence, they were assembled with the flow loop and gradually the fluid flow rate and pressures were increased until the values for the desired conditions were obtained. Further, the compliances, resistances and pulsatility were tuned to modify the shape of attained hemodynamic waveforms to simulate physiologic conditions. Pressure and flow were continually monitored throughout the duration of the experiment. For the ‘flow only’ condition, the valves, compliances and the collapsible chamber were not used.

HAECs were fixed with 4% paraformaldehyde in 1×PBS for 20 min, washed two times with wash buffer (1×PBS containing 0.05% Tween-20 (Fisher Scientific, Fair Lawn, N.J.)) and permeabilized with 0.5% Triton X-100 (Fisher Scientific, Fair Lawn, N.J.) for 2 min at room temperature. Then, cells were washed two times with wash buffer, blocked with 1% BSA in 1×PBS freshly prepared for 30 min and incubated with primary antibody anti-human mouse β-Catenin (1:50; Santa Cruz Biotechnology, Santa Cruz, Calif.) at room temperature for 1 h. Cells were washed three times with wash buffer for 5 min each time and incubated at room temperature with the second antibody, FITC-conjugated goat anti-mouse (1:100; Millipore, Billerica, Mass.). After 1 h, cells were washed three times with wash buffer. For negative controls, the same procedure was performed without adding the primary antibody. For the detection of F-actin, cells were washed two times with wash buffer, blocked with 1% BSA in 1×PBS for 30 min and incubated at room temperature for 1 h with TRITC-conjugated phalloidin (1:100; Millipore, Billerica, Mass.). Light Diagnostics mounting fluid (Millipore, Billerica, Mass.) was added to the cells and cells were examined using a Nikon Eclipse A1 Confocal Microscopy System (Nikon Instruments, Melville, N.Y.). Cell size was estimated using phase contrast microscopy in combination with fluorescent β-Catenin staining. Both phase contrast and fluorescence Images obtained at 40× magnification were overlaid to distinguish and map cell boundaries and analyzed using Metamorph Software (Molecular Devices, Sunnyvale, Calif.) to obtain the area of a cell. Measurements were made of 10 cells in each sample and the area was averaged.

A schematic of the device (and flow loop) is shown in FIG. 15A. The peristaltic pump flow rate determines the average levels of shear stress within the system. Culture of cells on a thin (500 μm) membrane allows generation physiological levels of stretch (10-25% constant strain and 5-10% cyclic strain) in response to applied pressure within the chamber. The primary component that introduces pulsatility or contractile function within the system is a pneumatically actuated collapsible chamber. The applied pressure, percentage systolic/diastolic fraction and frequency (beats per minute (bpm)) can be manipulated to alter frequency and amplitude of pressure and flow waveforms. In addition to this chamber, tunable compliance and flow resistance elements upstream of the inlet (pulmonary) and downstream of the outlet of the cell culture channel (aortic/systemic) allow modulation of flow resistance and modification of shape and amplitude of attained pressure and flow profiles. A one-way valve placed between the pulsatile chamber and the cell culture chamber ensures prevention of retrograde flows within the cell culture chamber.

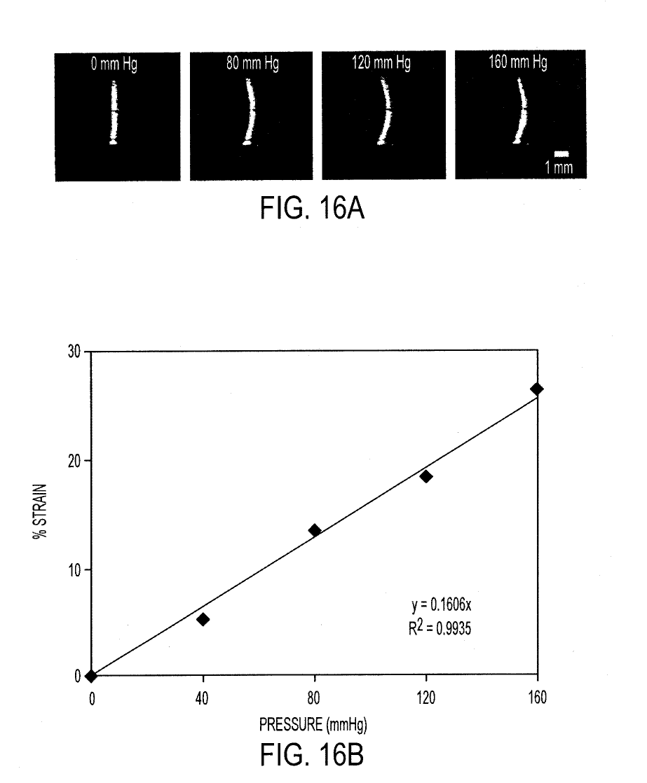

The 500 μm thick membrane within the ECCM system 400 fabricated out of PDMS (Young's Modulus˜500 KPa) was evaluated for strain as a function of applied pressure. Increase in pressure within the channel results in membrane stretch outwards. Hoop or circumferential strain that occurs in blood vessels is accurately replicated within this system and was verified using LIF. The dimensions of the channels (2.5 cm ‘l’ and 5 mm ‘w’) ensure that primary strain in the majority of the channel is hoop strain and is uniform along the channel except closer to the inlet and outlets. FIG. 16A shows the LIF images of the channel at 0, 80, 120, and 160 mmHg, respectively. FIG. 16B shows the strain distribution as a function of pressure. Strain is defined as the ratio of the elongation of the membrane and the original length when the applied gauge pressure is zero. As shown in the figure, the strain follows a linear trend. The field of view of the camera was 10.75 mm 4.80 mm yielding a nominal spatial resolution of 5.25 μm for the displacement data points. The camera views the intersection of the laser sheet and the channel at about 30° resulting in different resolutions in horizontal and vertical directions. The curvature of the back side of the channel (left side) is due to refraction of laser light through the curved front surface. When considering errors in image calibration and edge detection in the images, measurement of the PDMS surface profile is estimated to have an accuracy of ±5 μm in the physical plane.

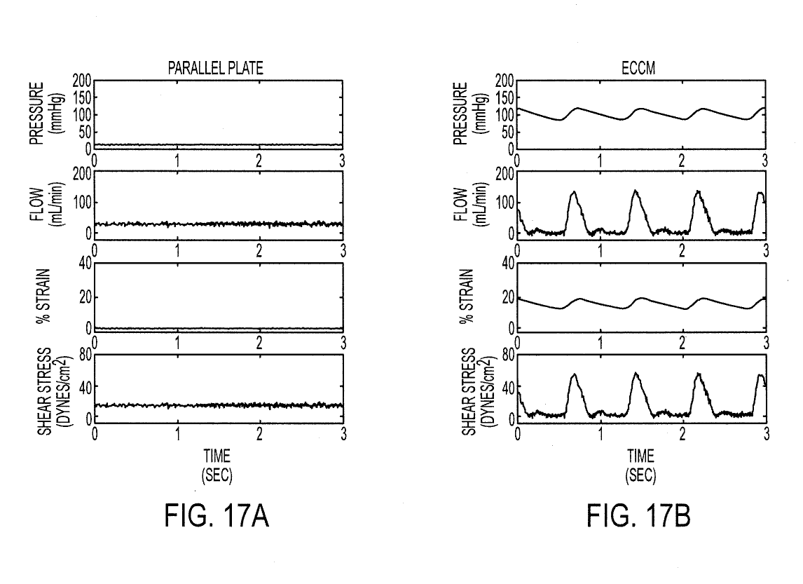

In an effort to compare and contrast the diversity in mechanical stress signals that are generated in the body and can be replicated with the ECCM system 400, pressure, flow, strain and shear stress profiles were compared using common parallel plate systems with the ECCM system 400 (FIGS. 17A, 17B). Parallel plate systems achieve constant laminar flow and shear stress but do not replicate pressure and stretch. Further in vivo stimulation is time varying due to the pulsatile nature of blood flow and causes pressure, flow, strain and shear stress values to vary between a minimum and maximum. The ECCM system not only achieves replication of realistic mechanical loading but also accurately mimics the dynamics of loading. The waveform morphologies achieved using this system corresponds very closely with clinical hemodynamic waveforms.

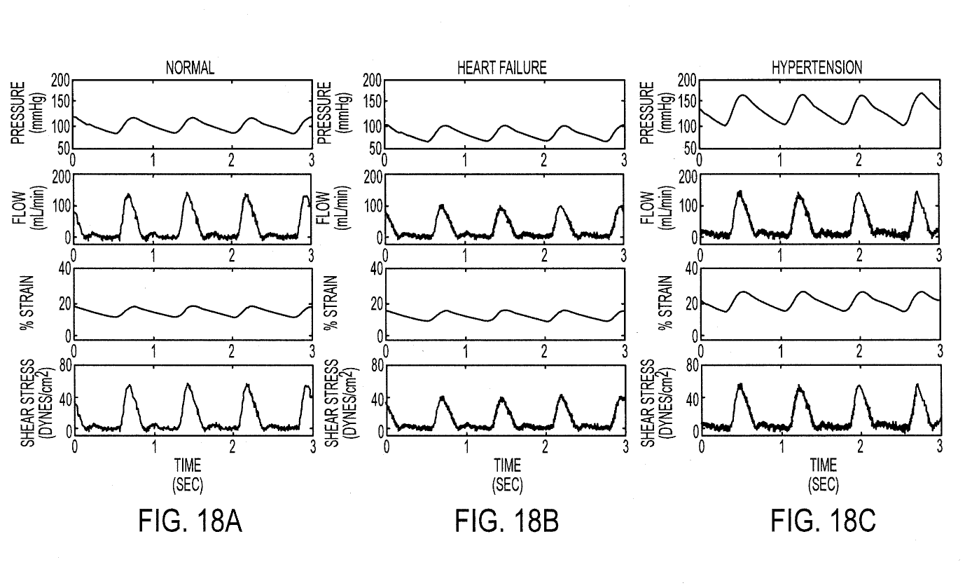

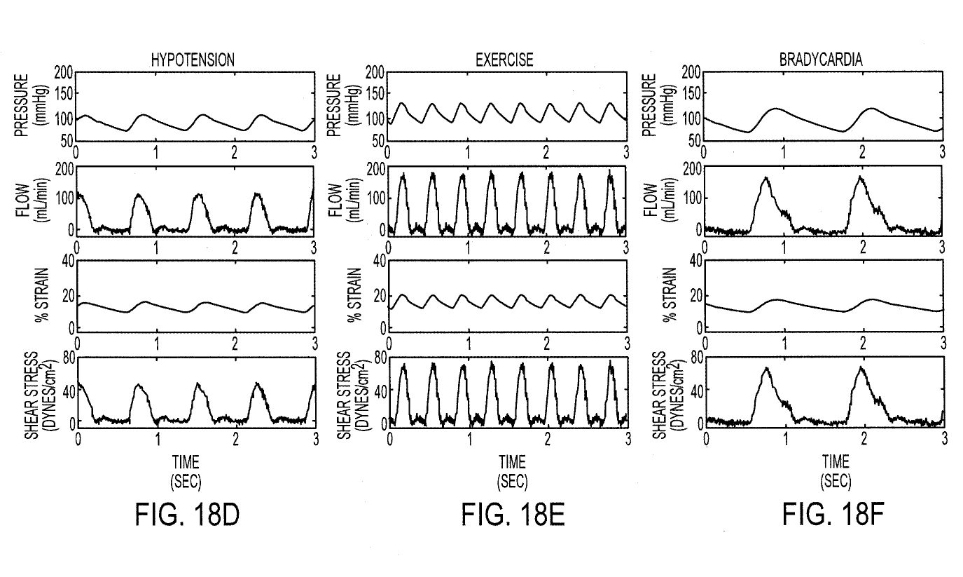

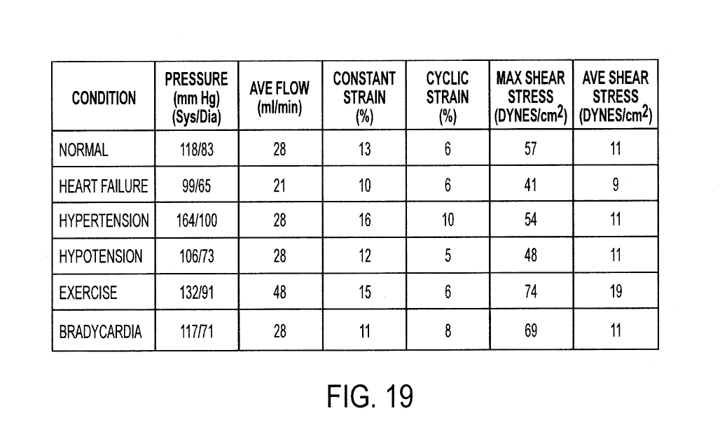

To demonstrate the capabilities of the ECCM system 400 to generate physiologically relevant conditions one or more conditions associated with normal and pathological conditions were simulated. (FIG. 18) (A) Normal, (B) Heart Failure, (C) Hypertension, (D) Hypotension, (E) Tachycardia/Exercise and (F) Bradycardia. For each of these conditions pressure, flow, strain and shear stress waveforms closely match clinically observed values. Critical values for each condition are also represented in FIG. 19. The normal condition represents pressure 118/83 mm Hg systolic/diastolic, 13% constant strain, 6% cyclic strain, average flow of 28 ml/min, and average shear stress of 11 dynes/cm². For the heart failure condition the compliance was decreased, arterial resistance was augmented and the average flow was decreased to 21 ml/min, which resulted in 99/65 mm Hg pressure, 10% constant strain, 6% cyclic strain and an average shear stress of 9 dynes/cm². Hypertension and hypotension conditions were established by maintaining average flows at 28 ml/min but the resistances and compliances were varied to generate pressures of 164/100 and 106/72 mm Hg. The average shear stress values remained at 11 dynes/cm² for both hyper- and hypotension while the constant strain values were at 16% and 12% with 10% and 5% cyclic strains, respectively. The exercise condition was obtained by increasing the frequency to 160 bpm, reducing arterial resistance and increasing the average flow rate to 48 ml/min resulting in pressures of 132/91, 15% constant strain, 6% cyclic strain and average shear stress 19 dynes/cm². Finally, the bradycardia condition was accomplished by setting the frequency at 40 bpm while the flow was maintained at 28 ml/min to achieve pressures of 117/71 mm Hg, constant strain 11%, cyclic strain 8% and average shear stress 11 dynes/cm².

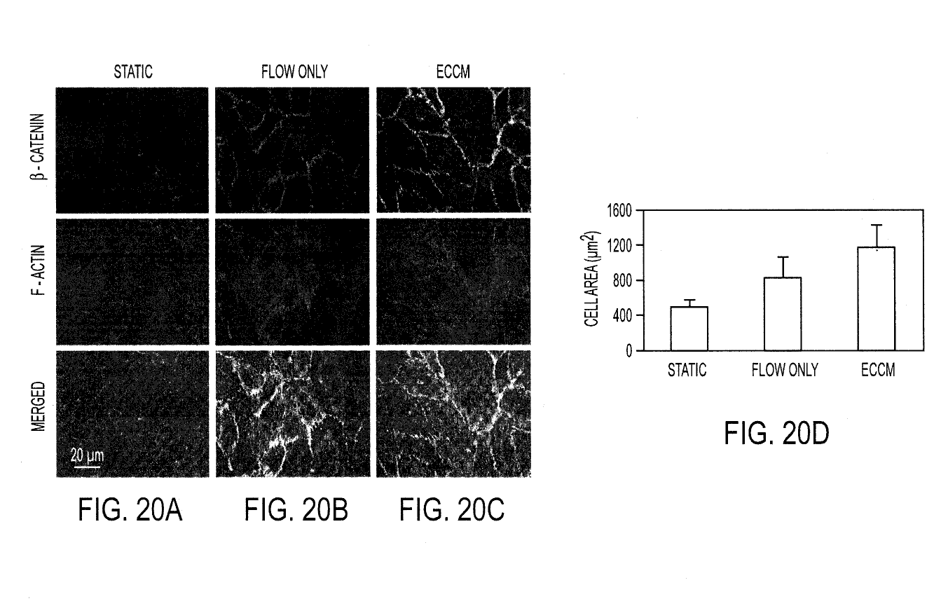

HAECs were cultured within the ECCM system 400 and maintained in culture for 24 hrs under normal conditions (Pressure ˜120/80 mm Hg systolic/diastolic, ˜13% constant strain and ˜6% variable strain, average shear stress of ˜11 dynes/cm²) and compared to ‘flow only’ (parallel plate, shear stress 15 dynes/cm²) and static controls. Following culture cells were examined using both phase contrast microscopy and confocal microscopy for alignment and morphology. HAECs cultured in the ECCM system 400 and ‘flow only’ control exhibited an aligned ellipsoidal phenotype in comparison to the static control in which cells were randomly oriented and polygonal in shape. Cytoskeletal alignment, cell attachment and cell-cell contacts were estimated using fluorescence microscopy using antibodies targeted against F-actin and β-Catenin (FIG. 20A-C). Overall cells within the ECCM system 400 were similar to ‘flow only’ controls and showed increased F-actin, alignment in the direction of flow and higher expression of cell adhesion molecule β-Catenin in comparison to static controls. HAECs were also evaluated for cell size using image analysis software and were found to be larger than both static and ‘flow only’ controls (FIG. 20D).

Cellular-level systems are useful in the study molecular signaling mechanisms involved in the normal functioning of healthy organs and tissue and more importantly changes that occur as a consequence of disease or injury. In vitro evaluation of isolated cells in the absence of connective tissue and other cells enables assessment of sub-cellular mechanisms in detail not possible using intact tissue preparations. However, this is accomplished at the expense of physiological relevance which is minimized due to the inability to fully recreate the physical and biochemical signaling environment in vitro. Blood vessels have a complex architecture where the innermost layer of ECs is surrounded by SMCs and fibroblasts. ECs are also constantly exposed to mechanical stimulation in the form of pulsatile pressure, stretch and shear due to hemodynamic loading which play critical roles in the maintenance of EC phenotype and function. Therefore, an important challenge in designing in vitro model systems is the ability to recreate critical aspects of the in vivo environment while maintaining the high level of specificity that is possible using isolated cell populations.

Despite efforts to replicate various aspects of in vivo mechanical loading in vitro, only a handful of groups have attempted replication of simultaneous pressure, stretch and shear loading. The most relevant models (FIG. 19) accomplish stimulation of cultured ECs with two or more mechanical stimuli. The preferred technique for EC culture in three of these models involves the use of distensible tubing and modulation of flow control. Culture within distensible tubing presents challenges in terms of seeding, imaging using confocal microscopy and extraction of cells from the tubing for subsequent analysis using standard laboratory techniques. Further, two of the three groups induce pulsatility within their systems by applying a time varying sinusoidal waveform to the pump flow rate which results in waveforms different from those observed in vivo. The third group offers the most accurate replication of pressure waveforms using a servo controlled pump with a feedback loop. However, this model is also associated with significant retrograde flow and both positive and negative shear stress values similar to oscillations seen in atherosclerosis susceptible regions. All of these groups do not incorporate a directional flow control valve (one-way) valve to prevent retrograde flows or multiple flow resistance and compliance elements within the system. These elements are important not only to mimic elements seen in vivo but also to accurately replicate pressure, flow, strain and shear stress waveforms seen in pathological conditions. The fourth group uses compliance and resistive elements and has shown ability to recreate pressure and shear waveforms associated with pathological conditions in blood vessels of different sizes but the shape of the waveforms are sinusoidal and the effect of stretch is completely ignored.

To overcome disadvantages associated with the previously mentioned models, the system 400 mimics in vivo pressure, flow, strain and shear stress waveforms we designed the ECCM system. The flow loop accounts for contractility, compliance, resistance and directional flow control and is integrated with a cell culture chamber to expose cells to mechanical loading. Multiple control elements tune the system to accurately replicate both normal and pathological conditions. Cell culture is accomplished on a thin polymeric membrane that can be integrated within a rectangular channel. This simplifies cell seeding and extraction using standard cell culture techniques and is compatible with confocal microscopy. The thin membrane on application of pressure stretches downward assuming a curved profile mimicking a section of the blood vessel. The amount of stretch can be controlled by varying the thickness of the membrane or adjusting the compliance of the chamber below the membrane by either varying the length of the air column or replacing the air with a different fluid. This can also be accomplished in a time varying fashion to represent dynamic changes in the ability of the membrane to stretch. A set of resistance and compliance elements prior to the inlet of the cell culture chamber represent the pulmonary compliance and resistance and also serve to eliminate the noise due to the peristaltic pump. Contractility (left ventricular function) is replicated using a pneumatically controlled collapsible chamber which can be tuned to represent changes in contractility, frequency and systolic fraction. A one way valve placed between the collapsible chamber and the cell culture chamber ensures elimination of retrograde flow. Another set of compliance and resistive elements downstream of the outlet of the cell culture chamber represent the aortic/systemic resistance and compliance.

Using this system, various conditions associated with normal and pathological conditions were replicated. These simulations match clinically observed waveforms. Transient negative values for flow and shear stress do not represent significant retrograde flow; rather they represent the closure of the one-way valve, which is very similar to what happens in vivo during closure of the aortic valve. The negative values are not sustained as can be seen from the flow and shear stress plots.

To demonstrate the ability of this system to culture relevant cellular populations, HAECs were seeded in the cell culture chamber and cultured under normal condition (pressure 120/80 mm Hg, 13% constant stretch, 6% cyclic stretch, 80 bpm and mean shear stress 11 dynes/cm²) and compared to ‘flow only’ (shear stress 13 dynes/cm²) and static controls. Results show an in vivo-like phenotype following culture in the ECCM system 400. Fluorescence microscopy shows similarities in cells cultured using ‘flow only’ and ECCM system 400. In comparison to static controls, cells cultured under both conditions exhibited an ellipsoidal shape, aligned cytoskeletal filaments and establishment of tight junctions as evidenced by increased expression of β-Catenin which plays an important role in maintaining a restrictive endothelial barrier. Cells in the ECCM system 400 also appear to be larger in size in comparison to both static and ‘flow only’ controls.

The ECCM system 400 enables design of experiments where physical loads can be dynamically manipulated. Cells cultured under normal conditions can be gradually or instantaneously exposed to loads associated with endothelial dysfunction causing changes in cell structure and function. Accomplishing this at the cellular level in vitro provides opportunities to probe in great detail the role of specific molecular mediators involved in the signaling associated with flow conditions seen in pathological conditions. Cells can be evaluated using microscopy directly within the μCCCM or cells or cellular contents can be extracted and evaluated for gene and protein expression. The cell culture medium can also be sampled continuously to monitor signaling through soluble factors. The ECCM system 400 can also be modified to reproduce flow patterns seen in atherosclerotic susceptible regions like branches and curves via removal of the directional flow control (one-way) valve and by lowering the flow rate to expose cells to oscillatory low shear stress flow. The diversity in conditions that can be replicated using the ECCM system 400 makes it an ideal platform to evaluate the effects of drugs and other adjunctive and conjunctive treatment options to deal with tissue damage, reorganization, accumulation of fatty deposits and to deal with local inflammation. The system 400 can be used to imitate any flow condition associated with normal or pathological cardiac flow states including normal, heart failure, hypertension, hypotension, exercise, tachycardia and bradycardia can be reproduced. In addition, waveforms associated with different vascular beds including arterial, arteriolar, capillary, venule, venous, pulmonary etc can be accurately reproduced, or waveforms associated with disease conditions like atherosclerosis in different regions of the arterial vessels can be reproduced. Still further, conditions like hypoxia, tissue injury, radiation can be replicated within the system. Endothelial cells, smooth muscle cells or co-cultures of different cells can be accomplished within the system. The system can be used to study ECs under normal, pathological conditions and also scenarios where the local flow behavior is affected by assist devices. In addition, the system 400 can be used to study, blood vessel damage, repair, regeneration and recovery.

The ECCM system 400 is used to culture tissue or cells, such as endothelial cells, using realistic in vivo-like pressure, flow, strain and shear stress waveforms. The system 400 uses various resistance, compliance and flow control elements in conjunction with a pump and a pneumatically actuated collapsible chamber to generate hemodynamic loading. The system 400 model was used to recreate various conditions associated with normal and pathological conditions. HAECs were cultured within this system to demonstrate attainment of an in vivo-like phenotype in comparison to static controls.

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present application. The scope of the invention should, therefore, be determined with reference to the appended claims, along with the full scope of equivalents to which such claims are entitled.

What is claimed is:

1. A system comprising:
a tissue/cell culture chamber including a tissue/cell culture membrane;
at least one collapsible valve fluidly coupled with the tissue/cell culture chamber;
a pump fluidly coupled with the tissue/cell culture chamber; and
a flow loop including the pump, chamber, and collapsible valve fluidly coupled together.

2. The system as recited in claim 1, further comprising at least one post adjacent the tissue/cell culture membrane.

3. The system as recited in claim 1, wherein the tissue/cell chamber is a channel, and the tissue/cell culture membrane forms a floor of the channel.

4. The system as recited in claim 1, further comprising a tunable hemostatic valve downstream of the collapsible valve.

5. The system as recited in claim 1, further comprising at least one pressure sensor in the tissue/cell culture chamber.

6. The system as recited in claim 1, wherein the collapsible valve is a tunable collapsible valve.

7. The system as recited in claim 1, further comprising cultured cells on tissue/cell culture the membrane.

8. The system as recited in claim 7, wherein the cultured cells are cardiomyocytes, smooth muscle cells or endothelial cells including cardiac, arterial, venous or pulmonary cells.

9. The system as recited in claim 1, further comprising compliance and resistance elements adapted to imitate pulmonary compliances and resistances, the tissue/cell culture chamber adapted to imitate conditions in a heart, a pulsatile chamber mimicking the heart, a one-way flow control valve, where the tissue/cell culture chamber, compliances and resistances elements adapted to imitate the systemic/aortic compliances and resistances.

10. The system as recited in claim 9, further comprising pressure and flow sensors.

11. The system as recited in claim 1, further comprising compliance and resistance elements upstream and downstream the collapsible valve.

12. A method comprising:
pumping fluid into a tissue/culture chamber with a pump;
closing a collapsible valve and raising pressure in the chamber; and
stretching a tissue/cell culture membrane, where the membrane has cultured cells.

13. The method as recited in claim 12, further comprising setting flow rates of the pump.

14. The method as recited in claim 12, further comprising increasing outflow resistance with a hemostatic valve downstream of the collapsible valve.

15. The method as recited in claim 12, further comprising predicting wall shear stress as a function of channel height and flow rate.

16. The method as recited in claim 12, further comprising modifying rate of flow with the pump.

17. The method as recited in claim 12, further comprising tuning one or more of collapsible volume, pressure used to achieve collapse, or frequency and timing of pressure of the collapsible valve.

18. The method as recited in claim 12, further comprising culturing cells on the tissue/cell culture membrane that allows cells to experience uniaxial or biaxial stretch.

19. The method as recited in claim 12, further comprising at least one compliance element and resistance element, using the compliance and resistance elements to imitate physiological or pathophysiological flow and pressure waveforms.

20. A system comprising:
a tissue/cell culture platform including at least a tissue/cell culture chamber;
a tissue/cell membrane having cultured cells thereon;
a pump fluidly coupled with multiple chamber tissue/cell culture platform;
a pressure generator communicatively coupled with tissue/cell culture platform; and
pressure sensitive valve fluidly coupled with the tissue/cell culture chamber that allows ejection of fluid from the tissue/cell culture chamber based on a predetermined pressure.

21. The system as recited in claim 20, further comprising a second membrane, the second membrane includes a post element coupled therewith, the post element having a rigidity greater than the second membrane.

22. A system comprising:
a flow loop including one or more tunable elements and a pump;
a tissue/cell culture chamber within the flow loop, the tissue/cell culture chamber having a tissue/cell membrane, the tissue/cell membrane having cultured cells thereon; the tissue/cell membrane is deformable in response to pressure buildup within the tissue/cell culture chamber and assumes a concave shape; and
a pulsatile chamber adapted to imitate a heart.

23. The system as recited in claim 22, wherein the tunable elements include compliance and resistance elements adapted to imitate pulmonary compliances and resistances.

24. The system as recited in claim 22, further comprising a one-way valve in the flow loop.

25. A method comprising:
introducing fluid into a tissue/cell culture chamber of a tissue/cell culture platform chamber system, the system including a tissue/cell culture platform including the tissue/cell culture chamber, a tissue/cell membrane having cultured cells thereon, a pump fluidly coupled with multiple chamber tissue/cell culture platform, a pressure generator communicatively coupled with tissue/cell culture platform, and a pressure sensitive valve fluidly coupled with the tissue/cell culture chamber that allows ejection of fluid from the tissue/cell culture chamber based on a predetermined pressure;
deflecting the tissue/cell membrane and stretching the cultured cells;
applying external pressure including compressing volume and increasing pressure in the tissue/cell culture chamber;
ejecting fluid from the tissue/cell culture chamber when pressure therein exceeds a predetermined value.

26. The method as recited in claim 25, wherein deflecting the tissue/cell membrane occurs without introducing pressure into the tissue/cell culture chamber.

27. The method as recited in claim 25, further comprising modulating at least one of pressure, stretch, flow or shear stress of the system.

28. The method as recited in claim 27, wherein modulating at least one or pressure, stretch, flow or shear stress includes stimulating the tissue/cell culture chamber with one or more pathological conditions similar to heart failure, hypotension, hypertension, tachycardia, or bradycardia.

29. The method as recited in claim 25, wherein the tissue/cell membrane includes at least one of tissue or cells thereon, the tissue/cells include one or more of smooth muscle cells, endothelial cells including arterial, venous, pulmonary, cardiac cells, or cardiomyocytes.
